(19) United States
(12) Patent Application Publication
(10) Pub. No.: US 2025/0243242 A1
(43) Pub. Date: Jul. 31, 2025

(54) LEVODOPA TYROSINE POLYMORPHS

(71) Applicant: NeuroDerm, Ltd., Rehovot (IL)

(72) Inventors: Avital Laxer, Rehovot (IL); Elana Gazal, Rehovot (IL); Alaa Talhami, Rehovot (IL)

(21) Appl. No.: 18/847,960

(22) PCT Filed: Mar. 16, 2023
(86) PCT No.: PCT/IL2023/050281

§ 371 Date: Sep. 17, 2024

Related U.S. Application Data
(60) Provisional application No. 63/321,202, filed on Mar. 18, 2022.

(51) Int. Cl. C07K 5/065; A61K 38/05

(52) U.S. Cl. CPC C07K 5/06078; A61K 38/05

(57) ABSTRACT

The disclosure is in part directed to crystalline forms of ((S)-2-amino-3-(3,4-dihydroxyphenyl)propanoyl)-A-tyrosine and pharmaceutical compositions thereof.

### CROSS REFERENCE

This application claims the benefit of and priority to U.S. Provisional Patent Application No. 63/321,202, filed Mar. 18, 2022, the disclosure of which is incorporated by reference herein in its entirety.

### BACKGROUND

Parkinson's disease is a degenerative condition characterized by reduced concentration of the neurotransmitter dopamine in the brain. Levodopa (L-dopa or L-3,4-dihydroxyphenylalanine) is an immediate metabolic precursor of dopamine that, unlike dopamine, is able to cross the blood brain barrier, and is most commonly used for restoring the dopamine concentration in the brain. Although levodopa has remained an effective therapy for the treatment of Parkinson's disease, some patients may become less responsive to levodopa, resulting in diminished benefits after about 3 or 4 years of therapy. Further, preparing liquid formulations of levodopa may be difficult. Thus, there remains a need for additional therapeutic agents for treating degenerative disorders such as Parkinson's disease.

Polymorphism is the ability of a substance to crystallize in more than one crystal lattice arrangement. Crystallization, or polymorphism, can influence many aspects of the solid-state properties of a drug substance. A crystalline substance may differ considerably from an amorphous form, and different crystal modifications of a substance may differ considerably from one another in many respects including solubility, dissolution rate and/or bioavailability. Generally, it is difficult to predict whether a given compound will form any crystalline solid-state forms. It is even more difficult to predict the physical properties of these crystalline solid-state forms. Therefore, it can be advantageous to have a crystalline form of a therapeutic agent for certain formulations and/or for manufacturing processes.

### SUMMARY

The present disclosure is directed, at least in part, to crystalline forms of ((S)-2-amino-3-(3,4-dihydroxyphenyl) propanoyl)-L-tyrosine, and crystalline anhydrates, hydrates and solvates thereof.

For example, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 20.1, for example, characterized by a powder X-ray diffraction pattern having characteristic peaks in degrees 2θ at about 12.6, 16.3, and 20.1, for example, characterized by a powder X-ray diffraction pattern having characteristic peaks in degrees 2θ at about 7.7, 8.7, 12.6, 16.3, 20.1, and 25.7, for example, characterized by a powder X-ray diffraction pattern having characteristic peaks in degrees 2θ at about 7.7, 8.7, 8.8, 12.6, 16.2, 16.3, 17.3, 20.1, 25.7, 29.8, 30.0, and 34.2.

((S)-2-amino-3-(3,4-dihydroxyphenyl) propanoyl)-L-tyrosine has the following chemical structure:

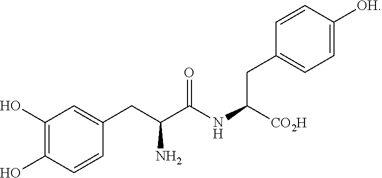 [SMILES: N[C@@H](Cc1ccc(O)c(O)c1)C(=O)N[C@@H](Cc1ccc(O)cc1)C(=O)O]

Further contemplated herein is a pharmaceutical composition comprising a disclosed crystalline form of ((S)-2-amino-3-(3,4-dihydroxyphenyl) propanoyl)-L-tyrosine and a pharmaceutically acceptable excipient. Further contemplated herein is a drug substance comprising at least a detectable amount of a disclosed form of ((S)-2-amino-3-(3,4-dihydroxyphenyl) propanoyl)-L-tyrosine. For example, disclosed herein is a drug substance comprising substantially pure crystalline form of ((S)-2-amino-3-(3,4-dihydroxyphenyl) propanoyl)-L-tyrosine.

### BRIEF DESCRIPTION OF THE DRAWINGS

FIG. 1 depicts the X-ray powder diffraction (XRPD) pattern of ((S)-2-amino-3-(3,4-dihydroxyphenyl) propanoyl)-L-tyrosine (Form N).

FIG. 2 depicts the differential scanning calorimetry (DSC) profile of Form N.

FIG. 3 depicts the thermogravimetric analysis (TGA) profile of Form N.

FIG. 4 depicts the X-ray powder diffraction (XRPD) patterns of ((S)-2-amino-3-(3,4-dihydroxyphenyl) propanoyl)-L-tyrosine Form A (top), Form B (middle), and Form C (bottom).

FIG. 5 depicts the differential scanning calorimetry (DSC) and

thermogravimetric analysis (TGA) profiles of Form B.

FIG. 6 depicts the differential scanning calorimetry (DSC) and thermogravimetric analysis (TGA) profiles of Form C.

FIG. 7 depicts the X-ray powder diffraction (XRPD) patterns of ((S)-2-amino-3-(3,4-dihydroxyphenyl) propanoyl)-L-tyrosine Form D (top), Form E (middle), and Form G (bottom).

FIG. 8 depicts the differential scanning calorimetry (DSC) and thermogravimetric analysis (TGA) profiles of Form D.

FIG. 9 depicts the differential scanning calorimetry (DSC) and thermogravimetric analysis (TGA) profiles of Form G.

FIG. 10 depicts the X-ray powder diffraction (XRPD) patterns of ((S)-2-amino-3-(3,4-dihydroxyphenyl) propanoyl)-L-tyrosine Form H (top), Form I (middle), and Form J (bottom).

FIG. 11 depicts the X-ray powder diffraction (XRPD) patterns of ((S)-2-amino-3-(3,4-dihydroxyphenyl) propanoyl)-L-tyrosine Form K (top), Form L (middle), and Form M (bottom).

FIG. 12 depicts the thermogravimetric analysis (TGA) profile of Form K.

FIG. 13 depicts the thermogravimetric analysis (TGA) profile of Form M.

FIG. 14 depicts the X-ray powder diffraction (XRPD) patterns of ((S)-2-amino-3-(3,4-dihydroxyphenyl) propanoyl)-L-tyrosine Form N (top) and Form O (bottom).

FIG. 15 depicts the X-ray powder diffraction (XRPD) patterns of ((S)-2-amino-3-(3,4-dihydroxyphenyl) propanoyl)-L-tyrosine Form P (top) and Form Q (bottom).

FIG. 16 depicts the X-ray powder diffraction (XRPD) pattern of amorphous ((S)-2-amino-3-(3,4-dihydroxyphenyl) propanoyl)-L-tyrosine.

FIG. 17 depicts the thermogravimetric analysis (TGA) profile of the amorphous form.

FIG. 18 depicts the differential scanning calorimetry (DSC) profile of the amorphous form.

FIG. 19 depicts the single X-ray crystal structure of crystalline Form N.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “crystalline form” refers to a crystal form or modification that can be characterized by analytical methods such as, e.g., X-ray powder diffraction (XRPD) and/or Differential scanning calorimetry (DSC). The crystalline compounds disclosed herein can exist in solvated as well as unsolvated forms with solvents such as water, ethanol, and the like. Unless otherwise indicated or inferred, it is intended that disclosed crystalline compounds include both solvated and unsolvated forms.

“Treating” includes any effect, e.g., lessening, reducing, modulating, or eliminating, that results in the improvement of the condition, disease, disorder and the like.

The term “disorder” refers to and is used interchangeably with, the terms “disease,” “condition,” or “illness,” unless otherwise indicated.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable excipient” or “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment, for example, of a cancer or a blood disorder is desired. “Modulation” includes antagonism (e.g., inhibition), agonism, partial antagonism and/or partial agonism.

In the present specification, the terms “effective amount” or “therapeutically effective amount” means the amount of the subject compound that will elicit the biological or medical response of a tissue, system or animal, (e.g. mammal or human) that is being sought by the researcher, veterinarian, medical doctor or other clinician. The compounds of the present disclosure are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of basic groups that may be present in compounds used in the compositions. Compounds included in the present compositions that are basic in nature are capable of forming a wide variety of salts with various inorganic and organic acids.

The term “and/or” is used in this disclosure to mean either “and” or “or” unless indicated otherwise.

As used herein, the words “a” and “an” are meant to include one or more unless otherwise specified. For example, the term “an agent” encompasses both a single agent and a combination of two or more agents.

Where the use of the term “about” is before a quantitative value, the present disclosure also includes the specific quantitative value itself, unless specifically stated otherwise. As used herein, the term “about” refers to a ±10% variation from the nominal value unless otherwise indicated or inferred. The term “about” in the context of peaks at degrees 2θ means that there is an uncertainty in the measurements of the 2θ of ±0.2 (expressed in 20), or that there is an uncertainty in the measurements of the 2θ of ±0.1 (expressed in 20), or that there is an uncertainty in the measurements of the 2θ of ±0.05 (expressed in 20). Generally, a DSC thermogram may have a variation in the range of ±3° C. Therefore, the temperature values should be understood as including values in the range of about +3° C.

In general, provided herein are crystalline forms of ((S)-2-amino-3-(3,4-dihydroxyphenyl) propanoyl)-L-tyrosine that are substantially free of any other crystalline forms, unless indicated otherwise. As used herein, “substantially pure”, “substantially free” or “substantially free of any other crystalline forms” means that the disclosed crystalline form contains about 20% or less, about 10% or less, about 5% or less, about 2% or less, or about 1% or less, of any other materials, e.g., other crystalline forms of ((S)-2-amino-3-(3,4-dihydroxyphenyl) propanoyl)-L-tyrosine as measured, for example, by XRPD, or less than about 20%, less than about 10%, less than about 5%, less than about 4%, less than about 3%, less than about 2% or less than about 1%, of any other materials, such as other crystalline forms of ((S)-2-amino-3-(3,4-dihydroxyphenyl) propanoyl)-L-tyrosine as measured, for example, by XRPD. Thus, a disclosed crystalline form of ((S)-2-amino-3-(3,4-dihydroxyphenyl) propanoyl)-L-tyrosine described herein as “substantially pure” or as “substantially free of any other crystalline forms” would be understood to contain greater than 80% (w/w), greater than 90% (w/w), greater than 95% (w/w), greater than 98% (w/w), or greater than 99% (w/w) of the said crystalline forms of ((S)-2-amino-3-(3,4-dihydroxyphenyl) propanoyl)-L-tyrosine. Accordingly, in some embodiments, a disclosed crystalline form of ((S)-2-amino-3-(3,4-dihydroxyphenyl) propanoyl)-L-tyrosine may contain from 1% to 20% (w/w), from 5% to 20% (w/w), or from 5% to 10% (w/w) of one or more other crystalline forms of ((S)-2-amino-3-(3,4-dihydroxyphenyl) propanoyl)-L-tyrosine.

### Crystalline Forms

The present disclosure is directed, at least in part, to crystalline forms of ((S)-2-amino-3-(3,4-dihydroxyphenyl) propanoyl)-L-tyrosine.

For example, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 20.1 (referred to herein as “Form N”).

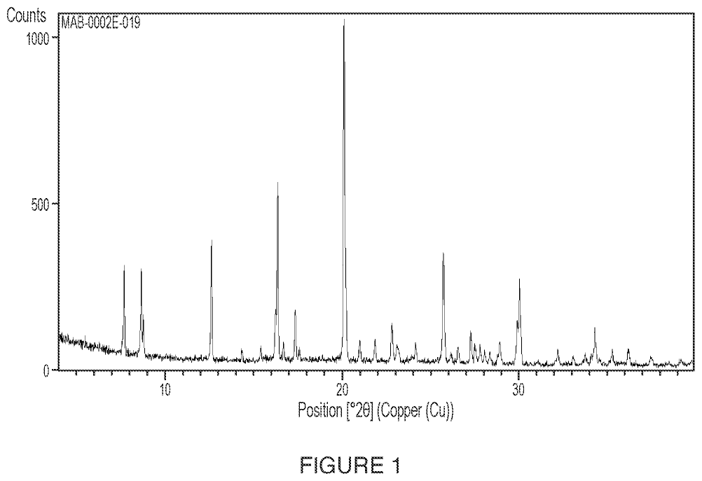

In one embodiment, the crystalline Form N of ((S)-2-amino-3-(3,4-dihydroxyphenyl) propanoyl)-L-tyrosine is characterized by a powder X-ray diffraction pattern that has a characteristic peak in degrees 2θ at about 7.7, is characterized by a powder X-ray diffraction pattern that has a characteristic peak in degrees 2θ at about 8.7, is characterized by a powder X-ray diffraction pattern that has a characteristic peak in degrees 2θ at about 8.8, is characterized by a powder X-ray diffraction pattern that has a characteristic peak in degrees 2θ at about 12.6, is characterized by a powder X-ray diffraction pattern that has a characteristic peak in degrees 2θ at about 16.2, is characterized by a powder X-ray diffraction pattern that has a characteristic peak in degrees 2θ at about 16.3, is characterized by a powder X-ray diffraction pattern that has a characteristic peak in degrees 2θ at about 17.3, is characterized by a powder X-ray diffraction pattern that has a characteristic peak in degrees 2θ at about 20.1, is characterized by a powder X-ray diffraction pattern that has a characteristic peak in degrees 2θ at about 25.7, is characterized by a powder X-ray diffraction pattern that has a characteristic peak in degrees 2θ at about 29.8, is characterized by a powder X-ray diffraction pattern that has a characteristic peak in degrees 2θ at about 30.0, and/or is characterized by a powder X-ray diffraction pattern that has a characteristic peak in degrees 2θ at about 34.2. In another embodiment, crystalline Form N is characterized by a powder X-ray diffraction pattern having at least one or more characteristic peaks in degrees 2θ at about 12.6, 16.3, and 20.1. In a further embodiment, crystalline Form N is characterized by a powder X-ray diffraction pattern having at least one or more characteristic peaks in degrees 2θ at about 7.7, 8.7, 12.6, 16.3, 20.1, and 25.7. In yet another embodiment, crystalline Form N is characterized by a powder X-ray diffraction pattern having at least one or more characteristic peaks in degrees 2θ at about 7.7, 8.7, 8.8, 12.6, 16.2, 16.3, 17.3, 20.1, 25.7, 29.8, 30.0, and 34.2. For example, a contemplated crystalline form has a powder X-ray diffraction pattern shown in FIG. 1. In one embodiment, the powder X-ray diffraction pattern of the crystalline form was obtained using Cu Kα radiation.

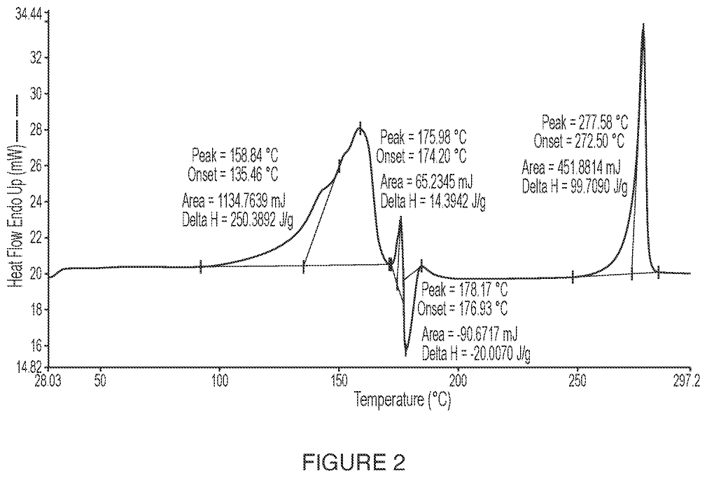

The contemplated crystalline Form N of ((S)-2-amino-3-(3,4-dihydroxyphenyl) propanoyl)-L-tyrosine may be characterized by a differential scanning calorimetry (DSC) profile showing a characteristic broad endotherm with an onset of about 135° C. and a peak of about 159° C.; a characteristic endotherm with an onset of about 174° C. and a peak of about 176° C.; a characteristic exotherm with an onset of about 177° C.; and a peak of about 178° C.; and a characteristic endotherm with an onset of about 272° C. and a peak of about 278° C. Form N, for example, may be characterized by a differential scanning calorimetry (DSC) profile showing an exothermic event with an onset of about 177° C. and a melting endotherm having an onset at about 272° C. Form N, for example, may be characterized by the differential scanning calorimetry profile shown in FIG. 2.

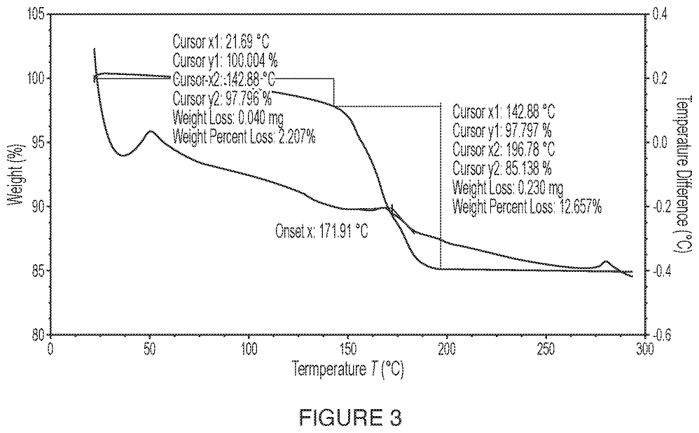

The contemplated crystalline Form N of ((S)-2-amino-3-(3,4-dihydroxyphenyl) propanoyl)-L-tyrosine may be characterized by a thermogravimetric analysis (TGA) profile showing a mass loss of about 15 wt. % between about 22° C. to about 196° C. For example, form N may be characterized by the TGA profile shown in FIG. 3. In some embodiments, crystalline Form N may be characterized by a dynamic vapor sorption (DVS) profile showing a reversable total mass change of about 0.6 wt. % between about 40 to about 80% relative humidity (RH) at 25° C. Crystalline Form N, for example, is physically stable at 75% RH and 40° C. In some embodiments, crystalline Form N may be characterized by Karl Fischer (KF) analysis showing 15.6% water by weight (about 3.7 equivalent by molar ratio).

The contemplated crystalline Form N of ((S)-2-amino-3-(3,4-dihydroxyphenyl) propanoyl)-L-tyrosine may be characterized by an aqueous solubility of about 184.34 mg/ml at pH of about 1.17, an aqueous solubility of about 9.46 mg/ml at pH of about 2.60, an aqueous solubility of about 4.06 mg/ml at pH of about 3.00, an aqueous solubility of about 2.86 mg/ml at of about pH 3.27, an aqueous solubility of about 1.54 mg/ml at pH of about 3.92, an aqueous solubility of about 1.64 mg/ml at pH of about 3.83, an aqueous solubility of about 1.33 mg/ml at pH of about 4.68, an aqueous solubility of about 1.29 mg/ml at pH of about 5.00, an aqueous solubility of about 1.37 mg/ml at pH of about 6.04, an aqueous solubility of about 0.51 mg/ml at pH of about 6.78, an aqueous solubility of about 18.86 mg/ml at pH of about 7.56, an aqueous solubility of about 111.38 mg/ml at pH of about 8.69, an aqueous solubility of about 58.68 mg/ml at pH of about 9.43, and/or an aqueous solubility of about 155.48 mg/ml at pH of about 10.26.

In another embodiment, disclosed herein is a crystalline form ((S)-2-amino-3-(3,4-dihydroxyphenyl) propanoyl)-L-tyrosine characterized by a powder X-ray diffraction pattern having a characteristic peak in degrees 2θ at about 19.0 (referred to herein as “Form A”).

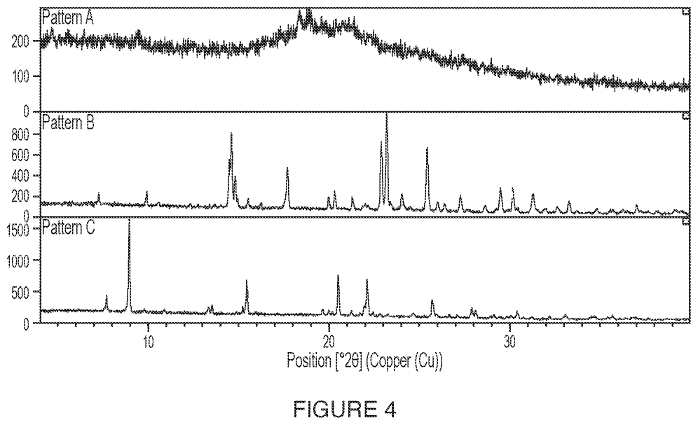

In one embodiment, the crystalline Form A of ((S)-2-amino-3-(3,4-dihydroxyphenyl) propanoyl)-L-tyrosine is characterized by a powder X-ray diffraction pattern that has a characteristic peak in degrees 2θ at about 4.7, is characterized by a powder X-ray diffraction pattern that has a characteristic peak in degrees 2θ at about 9.5, is characterized by a powder X-ray diffraction pattern that has a characteristic peak in degrees 2θ at about 18.3, is characterized by a powder X-ray diffraction pattern that has a characteristic peak in degrees 2θ at about 19.0, and/or is characterized by a powder X-ray diffraction pattern that has a characteristic peak in degrees 2θ at about 21.1. In another embodiment, crystalline Form A is characterized by a powder X-ray diffraction pattern having at least one or more characteristic peaks in degrees 2θ at about 4.7, 9.5, 18.3, 19.0, and 21.1. For example, a contemplated crystalline form has a powder X-ray diffraction pattern shown in FIG. 4. In one embodiment, the powder X-ray diffraction pattern of the crystalline form was obtained using Cu Kα radiation.

The contemplated crystalline Form A of ((S)-2-amino-3-(3,4-dihydroxyphenyl) propanoyl)-L-tyrosine may be characterized by a thermogravimetric analysis (TGA) profile showing a mass loss of about 10.2 wt. % between about 40° C. to about 200° C.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 23.2 (referred to herein as “Form B”).

In one embodiment, the crystalline Form B of ((S)-2-amino-3-(3,4-dihydroxyphenyl) propanoyl)-L-tyrosine is characterized by a powder X-ray diffraction pattern that has a characteristic peak in degrees 2θ at about 14.5, is characterized by a powder X-ray diffraction pattern that has a characteristic peak in degrees 2θ at about 14.6, is characterized by a powder X-ray diffraction pattern that has a characteristic peak in degrees 2θ at about 14.8, is characterized by a powder X-ray diffraction pattern that has a characteristic peak in degrees 2θ at about 17.7, is characterized by a powder X-ray diffraction pattern that has a characteristic peak in degrees 2θ at about 20.3, is characterized by a powder X-ray diffraction pattern that has a characteristic peak in degrees 2θ at about 22.9, is characterized by a powder X-ray diffraction pattern that has a characteristic peak in degrees 2θ at about 23.2, is characterized by a powder X-ray diffraction pattern that has a characteristic peak in degrees 2θ at about 25.4, is characterized by a powder X-ray diffraction pattern that has a characteristic peak in degrees 2θ at about 27.3, is characterized by a powder X-ray diffraction pattern that has a characteristic peak in degrees 2θ at about 29.5, is characterized by a powder X-ray diffraction pattern that has a characteristic peak in degrees 2θ at about 30.2, and/or is characterized by a powder X-ray diffraction pattern that has a characteristic peak in degrees 2θ at about 31.3. In another embodiment, crystalline Form B is characterized by a powder X-ray diffraction pattern having at least one or more characteristic peaks in degrees 2θ at about 14.5, 14.6, 14.8, 17.7, 20.3, 22.9, 23.2, 25.4, 27.3, 29.5, 30.2, and 31.3. For example, a contemplated crystalline form has a powder X-ray diffraction pattern shown in FIG. 4. In one embodiment, the powder X-ray diffraction pattern of the crystalline form was obtained using Cu Kα radiation.

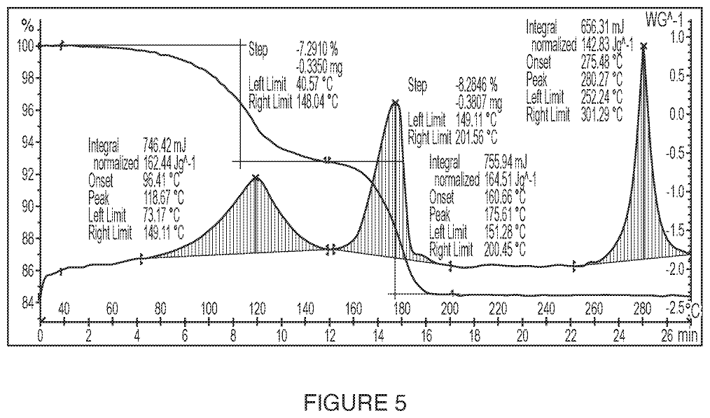

The contemplated crystalline Form B of ((S)-2-amino-3-(3,4-dihydroxyphenyl) propanoyl)-L-tyrosine may be characterized by a differential scanning calorimetry (DSC) profile showing a characteristic endotherm with an onset of about 96° C. and a peak of about 119° C.; a characteristic endotherm with an onset of about 161° C. and a peak of about 176° C.; and a characteristic endotherm with an onset of about 275° C. and a peak of about 280° C. Form B, for example, may be characterized by the differential scanning calorimetry profile shown in FIG. 5.

The contemplated crystalline Form B of ((S)-2-amino-3-(3,4-dihydroxyphenyl) propanoyl)-L-tyrosine may be characterized by a thermogravimetric analysis (TGA) profile showing a mass loss of about 15.6 wt. % between about 40° C. to about 200° C. (FIG. 5). In some embodiments, crystalline Form B may be characterized by Karl Fischer (KF) analysis showing 17.2% water by weight (about 2.2 equivalent by molar ratio).

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 9.0 (referred to herein as “Form C”).

In one embodiment, the crystalline Form C of ((S)-2-amino-3-(3,4-dihydroxyphenyl) propanoyl)-L-tyrosine is characterized by a powder X-ray diffraction pattern that has a characteristic peak in degrees 2θ at about 7.7, is characterized by a powder X-ray diffraction pattern that has a characteristic peak in degrees 2θ at about 9.0, is characterized by a powder X-ray diffraction pattern that has a characteristic peak in degrees 2θ at about 13.3, is characterized by a powder X-ray diffraction pattern that has a characteristic peak in degrees 2θ at about 13.5, is characterized by a powder X-ray diffraction pattern that has a characteristic peak in degrees 2θ at about 15.2, is characterized by a powder X-ray diffraction pattern that has a characteristic peak in degrees 2θ at about 15.4, is characterized by a powder X-ray diffraction pattern that has a characteristic peak in degrees 2θ at about 19.7, is characterized by a powder X-ray diffraction pattern that has a characteristic peak in degrees 2θ at about 20.5, is characterized by a powder X-ray diffraction pattern that has a characteristic peak in degrees 2θ at about 22.1, is characterized by a powder X-ray diffraction pattern that has a characteristic peak in degrees 2θ at about 25.7, is characterized by a powder X-ray diffraction pattern that has a characteristic peak in degrees 2θ at about 27.9, and/or is characterized by a powder X-ray diffraction pattern that has a characteristic peak in degrees 2θ at about 28.1. In another embodiment, crystalline Form C is characterized by a powder X-ray diffraction pattern having at least one or more characteristic peaks in degrees 2θ at about 7.7, 9.0, 13.3, 13.5, 15.2, 15.4, 19.7, 20.5, 22.1, 25.7, 27.9, and 28.1. For example, a contemplated crystalline form has a powder X-ray diffraction pattern shown in FIG. 4. In one embodiment, the powder X-ray diffraction pattern of the crystalline form was obtained using Cu Kα radiation.

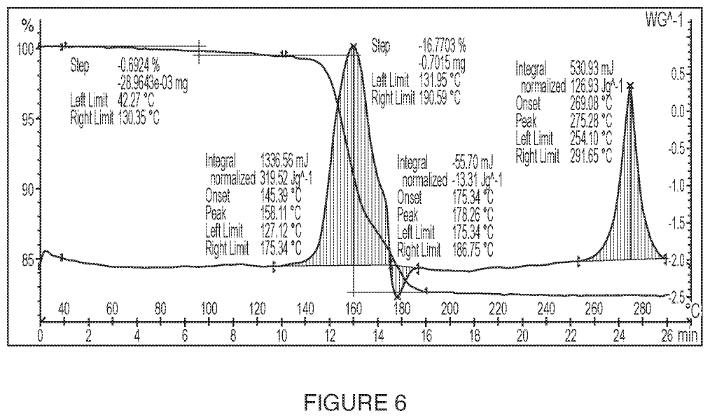

The contemplated crystalline Form C of ((S)-2-amino-3-(3,4-dihydroxyphenyl) propanoyl)-L-tyrosine may be characterized by a differential scanning calorimetry (DSC) profile showing a characteristic endotherm with an onset of about 145° C. and a peak of about 158° C.; a characteristic exotherm with an onset of about 175° C. and a peak of about 178° C.; and a characteristic endotherm with an onset of about 269° C. and a peak of about 275° C. (FIG. 6). Crystalline Form C of ((S)-2-amino-3-(3,4-dihydroxyphenyl) propanoyl)-L-tyrosine may be characterized by a thermogravimetric analysis (TGA) profile showing a mass loss of about 1.75 wt. % between about 30° C. to about 190° C. For example, Crystalline Form C may be characterized by a TGA profile as provided in FIG. 6.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 18.9 (referred to herein as “Form D”).

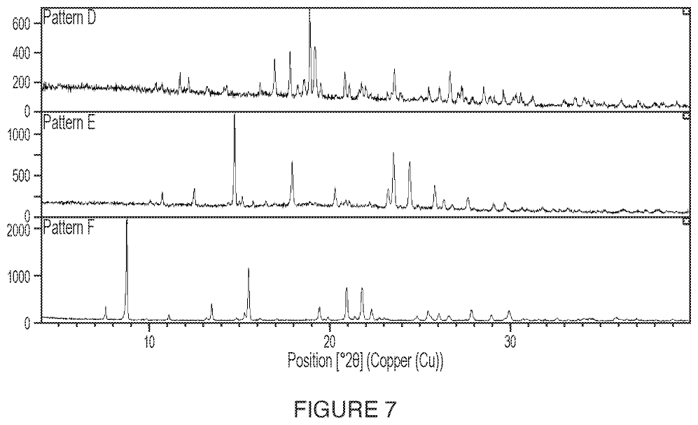

In one embodiment, the crystalline Form D of ((S)-2-amino-3-(3,4-dihydroxyphenyl) propanoyl)-L-tyrosine is characterized by a powder X-ray diffraction pattern that has a characteristic peak in degrees 2θ at about 11.7, is characterized by a powder X-ray diffraction pattern that has a characteristic peak in degrees 2θ at about 17.0, is characterized by a powder X-ray diffraction pattern that has a characteristic peak in degrees 2θ at about 17.8, is characterized by a powder X-ray diffraction pattern that has a characteristic peak in degrees 2θ at about 18.6, is characterized by a powder X-ray diffraction pattern that has a characteristic peak in degrees 2θ at about 18.9, is characterized by a powder X-ray diffraction pattern that has a characteristic peak in degrees 2θ at about 19.2, is characterized by a powder X-ray diffraction pattern that has a characteristic peak in degrees 2θ at about 20.9, is characterized by a powder X-ray diffraction pattern that has a characteristic peak in degrees 2θ at about 23.6, is characterized by a powder X-ray diffraction pattern that has a characteristic peak in degrees 2θ at about 26.1, is characterized by a powder X-ray diffraction pattern that has a characteristic peak in degrees 2θ at about 26.7, is characterized by a powder X-ray diffraction pattern that has a characteristic peak in degrees 2θ at about 27.3, and/or is characterized by a powder X-ray diffraction pattern that has a characteristic peak in degrees 2θ at about 28.5. In another embodiment, crystalline Form D is characterized by a powder X-ray diffraction pattern having at least one or more characteristic peaks in degrees 2θ at about 11.7, 17.0, 17.8, 18.6, 18.9, 19.2, 20.9, 23.6, 26.1, 26.7, 27.3, and 28.5. For example, a contemplated crystalline form has a powder X-ray diffraction pattern shown in FIG. 7. In one embodiment, the powder X-ray diffraction pattern of the crystalline form was obtained using Cu Kα radiation.

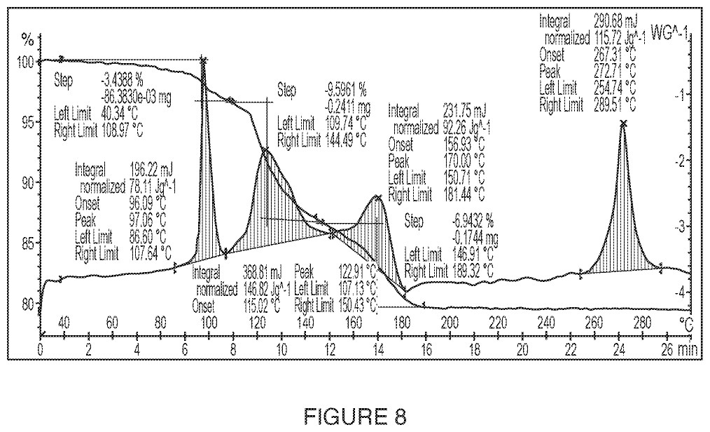

The contemplated crystalline Form D of ((S)-2-amino-3-(3,4-dihydroxyphenyl) propanoyl)-L-tyrosine may be characterized by a differential scanning calorimetry (DSC) profile showing a characteristic broad endotherm with an onset of about 79° C. and a peak of about 97° C.; a characteristic endotherm with an onset of about 147° C. and a peak of about 152° C.; a characteristic endotherm with an onset of about 175° C. and a peak of about 176° C.; a characteristic exotherm with an onset of about 178° C.; and a peak of about 179° C.; a characteristic exotherm with an onset of about 183° C. and a peak of about 191° C.; and a characteristic endotherm with an onset of about 263° C. and a peak of about 270° C. Form D, for example, may be characterized by the differential scanning calorimetry profile shown in FIG. 8. The contemplated crystalline Form D of ((S)-2-amino-3-(3,4-dihydroxyphenyl) propanoyl)-L-tyrosine may be characterized by a thermogravimetric analysis (TGA) profile showing a mass loss of about 38 wt. % between about 30° C. to about 211° C. For example, Crystalline Form D has a TGA profile as shown in FIG. 8.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 14.8 (referred to herein as “Form E”).

In one embodiment, the crystalline Form E of ((S)-2-amino-3-(3,4-dihydroxyphenyl) propanoyl)-L-tyrosine is characterized by a powder X-ray diffraction pattern that has a characteristic peak in degrees 2θ at about 10.8, is characterized by a powder X-ray diffraction pattern that has a characteristic peak in degrees 2θ at about 12.5, is characterized by a powder X-ray diffraction pattern that has a characteristic peak in degrees 2θ at about 14.8, is characterized by a powder X-ray diffraction pattern that has a characteristic peak in degrees 2θ at about 15.2, is characterized by a powder X-ray diffraction pattern that has a characteristic peak in degrees 2θ at about 17.9, is characterized by a powder X-ray diffraction pattern that has a characteristic peak in degrees 2θ at about 20.3, is characterized by a powder X-ray diffraction pattern that has a characteristic peak in degrees 2θ at about 20.9, is characterized by a powder X-ray diffraction pattern that has a characteristic peak in degrees 2θ at about 23.3, is characterized by a powder X-ray diffraction pattern that has a characteristic peak in degrees 2θ at about 23.5, is characterized by a powder X-ray diffraction pattern that has a characteristic peak in degrees 2θ at about 24.4, is characterized by a powder X-ray diffraction pattern that has a characteristic peak in degrees 2θ at about 25.8, and/or is characterized by a powder X-ray diffraction pattern that has a characteristic peak in degrees 2θ at about 26.3. In another embodiment, crystalline Form E is characterized by a powder X-ray diffraction pattern having at least one or more characteristic peaks in degrees 2θ at about 10.8, 12.5, 14.8, 15.2, 17.9, 20.3, 20.9, 23.3, 23.5, 24.4, 25.8, and 26.3. For example, a contemplated crystalline form has a powder X-ray diffraction pattern shown in FIG. 7. In one embodiment, the powder X-ray diffraction pattern of the crystalline form was obtained using Cu Kα radiation.

The contemplated crystalline Form E of ((S)-2-amino-3-(3,4-dihydroxyphenyl) propanoyl)-L-tyrosine may be characterized by a thermogravimetric analysis (TGA) profile showing a mass loss of about 11.5 wt. % between about 40° C. to about 200° C. In some embodiments, crystalline Form E may be characterized by a dynamic vapor sorption (DVS) profile showing a reversable total mass change of about 8 wt. % between about 0 to about 90% relative humidity (RH) at 25° C.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.8 (referred to herein as “Form G”).

In one embodiment, the crystalline Form G of ((S)-2-amino-3-(3,4-dihydroxyphenyl) propanoyl)-L-tyrosine is characterized by a powder X-ray diffraction pattern that has a characteristic peak in degrees 2θ at about 7.6, is characterized by a powder X-ray diffraction pattern that has a characteristic peak in degrees 2θ at about 8.8, is characterized by a powder X-ray diffraction pattern that has a characteristic peak in degrees 2θ at about 13.5, is characterized by a powder X-ray diffraction pattern that has a characteristic peak in degrees 2θ at about 15.3, is characterized by a powder X-ray diffraction pattern that has a characteristic peak in degrees 2θ at about 15.5, is characterized by a powder X-ray diffraction pattern that has a characteristic peak in degrees 2θ at about 19.4, is characterized by a powder X-ray diffraction pattern that has a characteristic peak in degrees 2θ at about 21.0, is characterized by a powder X-ray diffraction pattern that has a characteristic peak in degrees 2θ at about 21.8, is characterized by a powder X-ray diffraction pattern that has a characteristic peak in degrees 2θ at about 22.3, is characterized by a powder X-ray diffraction pattern that has a characteristic peak in degrees 2θ at about 25.4, is characterized by a powder X-ray diffraction pattern that has a characteristic peak in degrees 2θ at about 27.8, and/or is characterized by a powder X-ray diffraction pattern that has a characteristic peak in degrees 2θ at about 29.9. In another embodiment, crystalline Form G is characterized by a powder X-ray diffraction pattern having at least one or more characteristic peaks in degrees 2θ at about 7.6, 8.8, 13.5, 15.3, 15.5, 19.4, 21.0, 21.8, 22.3, 25.4, 27.8, and 29.9. For example, a contemplated crystalline form has a powder X-ray diffraction pattern shown in FIG. 7. In one embodiment, the powder X-ray diffraction pattern of the crystalline form was obtained using Cu Kα radiation.

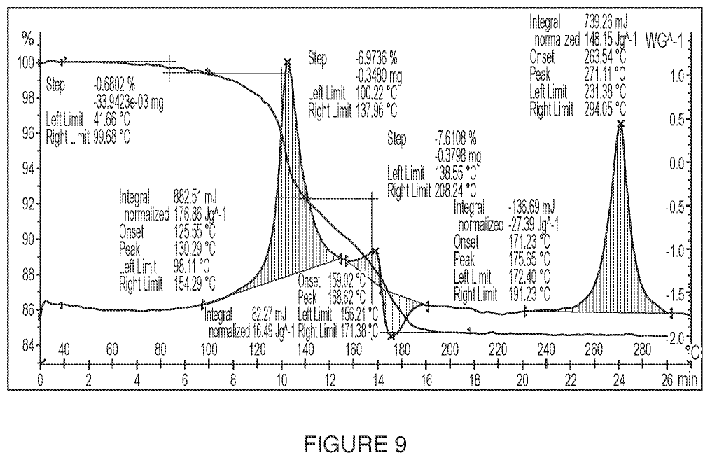

The contemplated crystalline Form G of ((S)-2-amino-3-(3,4-dihydroxyphenyl) propanoyl)-L-tyrosine may be characterized by a differential scanning calorimetry (DSC) profile showing a characteristic endotherm with an onset of about 126° C. and a peak of about 130° C.; a characteristic endotherm with an onset of about 159° C. and a peak of about 169° C.; a characteristic exotherm with an onset of about 171° C.; and a peak of about 176° C.; and a characteristic endotherm with an onset of about 264° C. and a peak of about 271° C. Form G, for example, may be characterized by the differential scanning calorimetry profile shown in FIG. 9.

The contemplated crystalline Form G of ((S)-2-amino-3-(3,4-dihydroxyphenyl) propanoyl)-L-tyrosine may be characterized by a thermogravimetric analysis (TGA) profile showing a mass loss of about 15 wt. % between about 42° C. to about 208° C. (FIG. 9). In some embodiments, crystalline Form G may be characterized by a dynamic vapor sorption (DVS) profile showing a reversable total mass change of about 2.6 wt. % between about 40 to about 80% relative humidity (RH) at 25° C. In some embodiments, crystalline Form G may be characterized by Karl Fischer (KF) analysis showing 12.3% water by weight (about 2.3 equivalent by molar ratio).

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.7 (referred to herein as “Form H”).

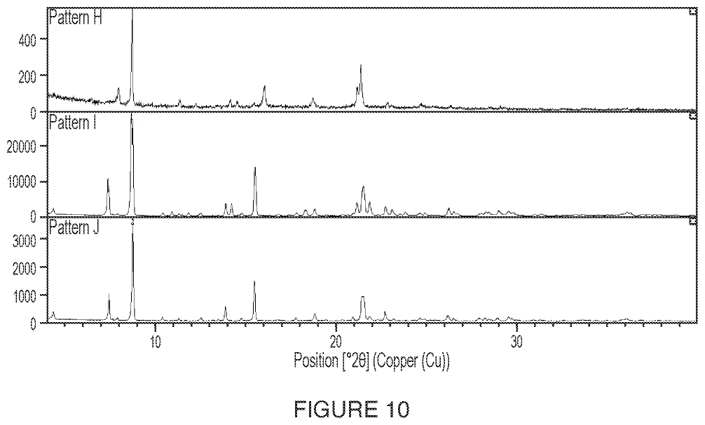

In one embodiment, the crystalline Form H of ((S)-2-amino-3-(3,4-dihydroxyphenyl) propanoyl)-L-tyrosine is characterized by a powder X-ray diffraction pattern that has a characteristic peak in degrees 2θ at about 8.0, is characterized by a powder X-ray diffraction pattern that has a characteristic peak in degrees 2θ at about 8.7, is characterized by a powder X-ray diffraction pattern that has a characteristic peak in degrees 2θ at about 11.4, is characterized by a powder X-ray diffraction pattern that has a characteristic peak in degrees 2θ at about 12.3, is characterized by a powder X-ray diffraction pattern that has a characteristic peak in degrees 2θ at about 14.2, is characterized by a powder X-ray diffraction pattern that has a characteristic peak in degrees 2θ at about 14.6, is characterized by a powder X-ray diffraction pattern that has a characteristic peak in degrees 2θ at about 15.5, is characterized by a powder X-ray diffraction pattern that has a characteristic peak in degrees 2θ at about 16.1, is characterized by a powder X-ray diffraction pattern that has a characteristic peak in degrees 2θ at about 18.7, is characterized by a powder X-ray diffraction pattern that has a characteristic peak in degrees 2θ at about 21.1, is characterized by a powder X-ray diffraction pattern that has a characteristic peak in degrees 2θ at about 21.4, and/or is characterized by a powder X-ray diffraction pattern that has a characteristic peak in degrees 2θ at about 22.9. In another embodiment, crystalline Form H is characterized by a powder X-ray diffraction pattern having at least one or more characteristic peaks in degrees 2θ at about 8.0, 8.7, 11.4, 12.3, 14.2, 14.6, 15.5, 16.1, 18.7, 21.1, 21.4, and 22.9. For example, a contemplated crystalline form has a powder X-ray diffraction pattern shown in FIG. 10. In one embodiment, the powder X-ray diffraction pattern of the crystalline form was obtained using Cu Kα radiation.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.7 (referred to herein as “Form I”).

In one embodiment, the crystalline Form I of ((S)-2-amino-3-(3,4-dihydroxyphenyl) propanoyl)-L-tyrosine is characterized by a powder X-ray diffraction pattern that has a characteristic peak in degrees 2θ at about 7.4, is characterized by a powder X-ray diffraction pattern that has a characteristic peak in degrees 2θ at about 7.5, is characterized by a powder X-ray diffraction pattern that has a characteristic peak in degrees 2θ at about 8.7, is characterized by a powder X-ray diffraction pattern that has a characteristic peak in degrees 2θ at about 8.8, is characterized by a powder X-ray diffraction pattern that has a characteristic peak in degrees 2θ at about 13.9, is characterized by a powder X-ray diffraction pattern that has a characteristic peak in degrees 2θ at about 14.2, is characterized by a powder X-ray diffraction pattern that has a characteristic peak in degrees 2θ at about 15.5, is characterized by a powder X-ray diffraction pattern that has a characteristic peak in degrees 2θ at about 21.1, is characterized by a powder X-ray diffraction pattern that has a characteristic peak in degrees 2θ at about 21.6, is characterized by a powder X-ray diffraction pattern that has a characteristic peak in degrees 2θ at about 21.9, is characterized by a powder X-ray diffraction pattern that has a characteristic peak in degrees 2θ at about 22.7, and/or is characterized by a powder X-ray diffraction pattern that has a characteristic peak in degrees 2θ at about 26.2. In another embodiment, crystalline Form I is characterized by a powder X-ray diffraction pattern having at least one or more characteristic peaks in degrees 2θ at about 7.4, 7.5, 8.7, 8.8, 13.9, 14.2, 15.5, 21.1, 21.6, 21.9, 22.7, and 26.2. For example, a contemplated crystalline form has a powder X-ray diffraction pattern shown in FIG. 10. In one embodiment, the powder X-ray diffraction pattern of the crystalline form was obtained using Cu Kα radiation.

The contemplated crystalline Form I of ((S)-2-amino-3-(3,4-dihydroxyphenyl) propanoyl)-L-tyrosine may be characterized by a thermogravimetric analysis (TGA) profile showing a mass loss of about 11 wt. % between about 30° C. to about 212° C.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.8 (referred to herein as “Form J”).

In one embodiment, the crystalline Form J of ((S)-2-amino-3-(3,4-dihydroxyphenyl) propanoyl)-L-tyrosine is characterized by a powder X-ray diffraction pattern that has a characteristic peak in degrees 2θ at about 4.4, is characterized by a powder X-ray diffraction pattern that has a characteristic peak in degrees 2θ at about 7.5, is characterized by a powder X-ray diffraction pattern that has a characteristic peak in degrees 2θ at about 8.8, is characterized by a powder X-ray diffraction pattern that has a characteristic peak in degrees 2θ at about 13.9, is characterized by a powder X-ray diffraction pattern that has a characteristic peak in degrees 2θ at about 15.5, is characterized by a powder X-ray diffraction pattern that has a characteristic peak in degrees 2θ at about 18.9, is characterized by a powder X-ray diffraction pattern that has a characteristic peak in degrees 2θ at about 21.4, is characterized by a powder X-ray diffraction pattern that has a characteristic peak in degrees 2θ at about 21.6, is characterized by a powder X-ray diffraction pattern that has a characteristic peak in degrees 2θ at about 21.8, is characterized by a powder X-ray diffraction pattern that has a characteristic peak in degrees 2θ at about 22.7, is characterized by a powder X-ray diffraction pattern that has a characteristic peak in degrees 2θ at about 26.2, and/or is characterized by a powder X-ray diffraction pattern that has a characteristic peak in degrees 2θ at about 29.5. In another embodiment, crystalline Form J is characterized by a powder X-ray diffraction pattern having at least one or more characteristic peaks in degrees 2θ at about 4.4, 7.5, 8.8, 13.9, 15.5, 18.9, 21.4, 21.6, 21.8, 22.7, 26.2, and 29.5. For example, a contemplated crystalline form has a powder X-ray diffraction pattern shown in FIG. 10. In one embodiment, the powder X-ray diffraction pattern of the crystalline form was obtained using Cu Kα radiation.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 15.4 (referred to herein as “Form K”).

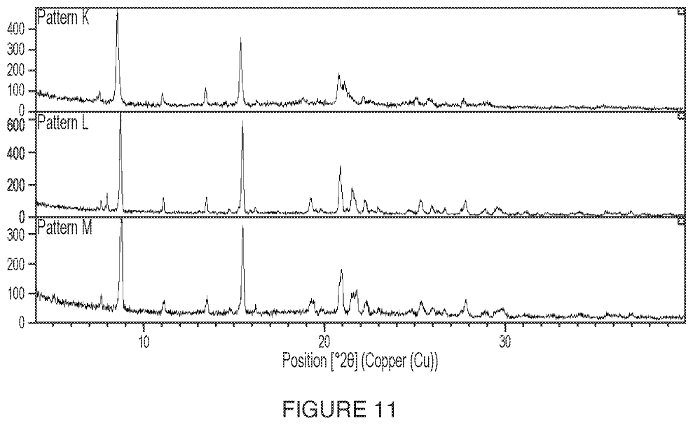

In one embodiment, the crystalline Form K of ((S)-2-amino-3-(3,4-dihydroxyphenyl) propanoyl)-L-tyrosine is characterized by a powder X-ray diffraction pattern that has a characteristic peak in degrees 2θ at about 7.5, is characterized by a powder X-ray diffraction pattern that has a characteristic peak in degrees 2θ at about 8.5, is characterized by a powder X-ray diffraction pattern that has a characteristic peak in degrees 2θ at about 11.0, is characterized by a powder X-ray diffraction pattern that has a characteristic peak in degrees 2θ at about 13.4, is characterized by a powder X-ray diffraction pattern that has a characteristic peak in degrees 2θ at about 15.4, is characterized by a powder X-ray diffraction pattern that has a characteristic peak in degrees 2θ at about 18.8, is characterized by a powder X-ray diffraction pattern that has a characteristic peak in degrees 2θ at about 20.8, is characterized by a powder X-ray diffraction pattern that has a characteristic peak in degrees 2θ at about 21.0, is characterized by a powder X-ray diffraction pattern that has a characteristic peak in degrees 2θ at about 22.1, is characterized by a powder X-ray diffraction pattern that has a characteristic peak in degrees 2θ at about 25.1, is characterized by a powder X-ray diffraction pattern that has a characteristic peak in degrees 2θ at about 25.8, and/or is characterized by a powder X-ray diffraction pattern that has a characteristic peak in degrees 2θ at about 27.7. In another embodiment, crystalline Form K is characterized by a powder X-ray diffraction pattern having at least one or more characteristic peaks in degrees 2θ at about 7.5, 8.5, 11.0, 13.4, 15.4, 18.8, 20.8, 21.0, 22.1, 25.1, 25.8, and 27.7. For example, a contemplated crystalline form has a powder X-ray diffraction pattern shown in FIG. 11. In one embodiment, the powder X-ray diffraction pattern of the crystalline form was obtained using Cu Kα radiation.

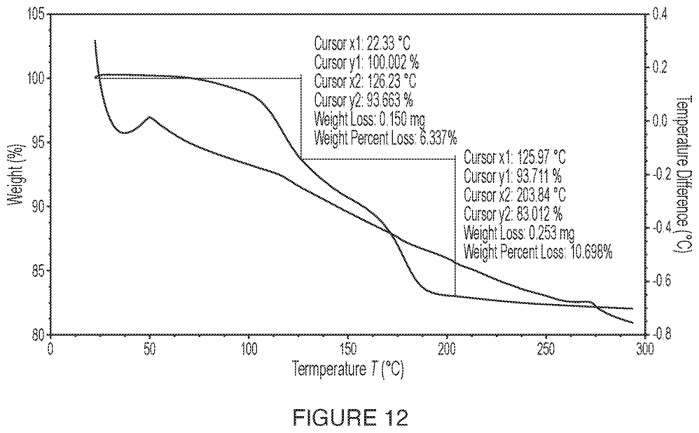

The contemplated crystalline Form K of ((S)-2-amino-3-(3,4-dihydroxyphenyl) propanoyl)-L-tyrosine may be characterized by a thermogravimetric analysis (TGA) profile showing a mass loss of about 17 wt. % between about 22° C. to about 203° C. (FIG. 12).

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.7 (referred to herein as “Form L”).

In one embodiment, the crystalline Form L of ((S)-2-amino-3-(3,4-dihydroxyphenyl) propanoyl)-L-tyrosine is characterized by a powder X-ray diffraction pattern that has a characteristic peak in degrees 2θ at about 7.6, is characterized by a powder X-ray diffraction pattern that has a characteristic peak in degrees 2θ at about 8.0, is characterized by a powder X-ray diffraction pattern that has a characteristic peak in degrees 2θ at about 8.7, is characterized by a powder X-ray diffraction pattern that has a characteristic peak in degrees 2θ at about 11.1, is characterized by a powder X-ray diffraction pattern that has a characteristic peak in degrees 2θ at about 13.4, is characterized by a powder X-ray diffraction pattern that has a characteristic peak in degrees 2θ at about 15.5, is characterized by a powder X-ray diffraction pattern that has a characteristic peak in degrees 2θ at about 19.2, is characterized by a powder X-ray diffraction pattern that has a characteristic peak in degrees 2θ at about 20.9, is characterized by a powder X-ray diffraction pattern that has a characteristic peak in degrees 2θ at about 21.5, is characterized by a powder X-ray diffraction pattern that has a characteristic peak in degrees 2θ at about 22.2, is characterized by a powder X-ray diffraction pattern that has a characteristic peak in degrees 2θ at about 25.3, and/or is characterized by a powder X-ray diffraction pattern that has a characteristic peak in degrees 2θ at about 27.8. In another embodiment, crystalline Form L is characterized by a powder X-ray diffraction pattern having at least one or more characteristic peaks in degrees 2θ at about 7.6, 8.0, 8.7, 11.1, 13.4, 15.5, 19.2, 20.9, 21.5, 22.2, 25.3, and 27.8. For example, a contemplated crystalline form has a powder X-ray diffraction pattern shown in FIG. 11. In one embodiment, the powder X-ray diffraction pattern of the crystalline form was obtained using Cu Kα radiation.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.7 (referred to herein as “Form M”).

In one embodiment, the crystalline Form M of ((S)-2-amino-3-(3,4-dihydroxyphenyl) propanoyl)-L-tyrosine is characterized by a powder X-ray diffraction pattern that has a characteristic peak in degrees 2θ at about 7.6, is characterized by a powder X-ray diffraction pattern that has a characteristic peak in degrees 2θ at about 8.7, is characterized by a powder X-ray diffraction pattern that has a characteristic peak in degrees 2θ at about 8.8, is characterized by a powder X-ray diffraction pattern that has a characteristic peak in degrees 2θ at about 11.1, is characterized by a powder X-ray diffraction pattern that has a characteristic peak in degrees 2θ at about 13.5, is characterized by a powder X-ray diffraction pattern that has a characteristic peak in degrees 2θ at about 15.5, is characterized by a powder X-ray diffraction pattern that has a characteristic peak in degrees 2θ at about 20.9, is characterized by a powder X-ray diffraction pattern that has a characteristic peak in degrees 2θ at about 21.5, is characterized by a powder X-ray diffraction pattern that has a characteristic peak in degrees 2θ at about 21.8, is characterized by a powder X-ray diffraction pattern that has a characteristic peak in degrees 2θ at about 22.3, is characterized by a powder X-ray diffraction pattern that has a characteristic peak in degrees 2θ at about 25.3, and/or is characterized by a powder X-ray diffraction pattern that has a characteristic peak in degrees 2θ at about 27.8. In another embodiment, crystalline Form M is characterized by a powder X-ray diffraction pattern having at least one or more characteristic peaks in degrees 2θ at about 7.6, 8.7, 8.8, 11.1, 13.5, 15.5, 20.9, 21.5, 21.8, 22.3, 25.3, and 27.8. For example, a contemplated crystalline form has a powder X-ray diffraction pattern shown in FIG. 11. In one embodiment, the powder X-ray diffraction pattern of the crystalline form was obtained using Cu Kα radiation.

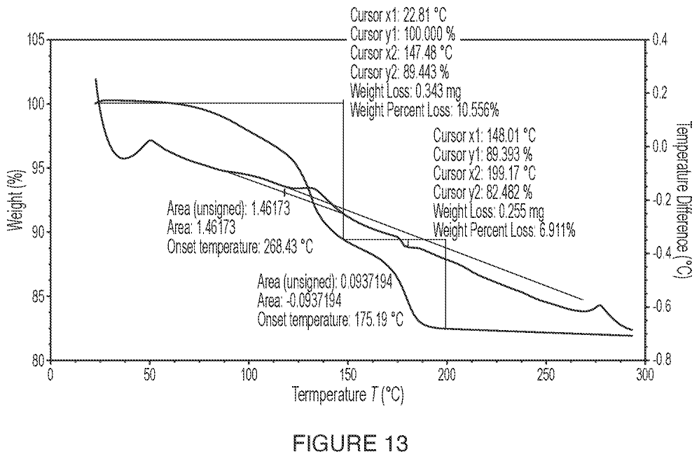

The contemplated crystalline Form M of ((S)-2-amino-3-(3,4-dihydroxyphenyl) propanoyl)-L-tyrosine may be characterized by a thermogravimetric analysis (TGA) profile showing a mass loss of about 17 wt. % between about 22° C. to about 199° C. For example, a Crystalline Form M may have a TGA profile as shown in FIG. 13. In some embodiments, crystalline Form M may be characterized by Karl Fischer (KF) analysis showing 17.8% water by weight (about 4.3 equivalent by molar ratio).

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.7 (referred to herein as “Form O”).

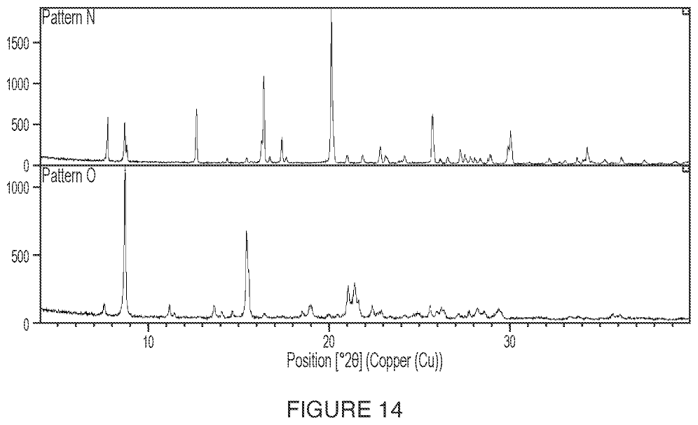

In one embodiment, the crystalline Form O of ((S)-2-amino-3-(3,4-dihydroxyphenyl) propanoyl)-L-tyrosine is characterized by a powder X-ray diffraction pattern that has a characteristic peak in degrees 2θ at about 7.5, is characterized by a powder X-ray diffraction pattern that has a characteristic peak in degrees 2θ at about 8.7, is characterized by a powder X-ray diffraction pattern that has a characteristic peak in degrees 2θ at about 11.1, is characterized by a powder X-ray diffraction pattern that has a characteristic peak in degrees 2θ at about 13.6, is characterized by a powder X-ray diffraction pattern that has a characteristic peak in degrees 2θ at about 15.4, is characterized by a powder X-ray diffraction pattern that has a characteristic peak in degrees 2θ at about 15.5, is characterized by a powder X-ray diffraction pattern that has a characteristic peak in degrees 2θ at about 18.9, is characterized by a powder X-ray diffraction pattern that has a characteristic peak in degrees 2θ at about 21.0, is characterized by a powder X-ray diffraction pattern that has a characteristic peak in degrees 2θ at about 21.4, is characterized by a powder X-ray diffraction pattern that has a characteristic peak in degrees 2θ at about 21.6, is characterized by a powder X-ray diffraction pattern that has a characteristic peak in degrees 2θ at about 22.3, and/or is characterized by a powder X-ray diffraction pattern that has a characteristic peak in degrees 2θ at about 25.5. In another embodiment, crystalline Form O is characterized by a powder X-ray diffraction pattern having at least one or more characteristic peaks in degrees 2θ at about 7.5, 8.7, 11.1, 13.6, 15.4, 15.5, 18.9, 21.0, 21.4, 21.6, 22.3, and 25.5. For example, a contemplated crystalline form has a powder X-ray diffraction pattern shown in FIG. 14. In one embodiment, the powder X-ray diffraction pattern of the crystalline form was obtained using Cu Kα radiation.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.7 (referred to herein as “Form P”).

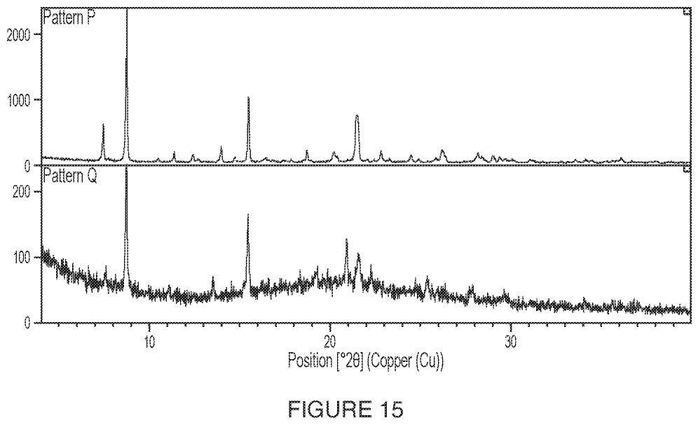

In one embodiment, the crystalline Form P of ((S)-2-amino-3-(3,4-dihydroxyphenyl) propanoyl)-L-tyrosine is characterized by a powder X-ray diffraction pattern that has a characteristic peak in degrees 2θ at about 7.4, is characterized by a powder X-ray diffraction pattern that has a characteristic peak in degrees 2θ at about 8.7, is characterized by a powder X-ray diffraction pattern that has a characteristic peak in degrees 2θ at about 11.4, is characterized by a powder X-ray diffraction pattern that has a characteristic peak in degrees 2θ at about 14.0, is characterized by a powder X-ray diffraction pattern that has a characteristic peak in degrees 2θ at about 15.5, is characterized by a powder X-ray diffraction pattern that has a characteristic peak in degrees 2θ at about 18.7, is characterized by a powder X-ray diffraction pattern that has a characteristic peak in degrees 2θ at about 20.2, is characterized by a powder X-ray diffraction pattern that has a characteristic peak in degrees 2θ at about 21.4, is characterized by a powder X-ray diffraction pattern that has a characteristic peak in degrees 2θ at about 21.5, is characterized by a powder X-ray diffraction pattern that has a characteristic peak in degrees 2θ at about 22.8, is characterized by a powder X-ray diffraction pattern that has a characteristic peak in degrees 2θ at about 26.2, and/or is characterized by a powder X-ray diffraction pattern that has a characteristic peak in degrees 2θ at about 28.2. In another embodiment, crystalline Form P is characterized by a powder X-ray diffraction pattern having at least one or more characteristic peaks in degrees 2θ at about 7.4, 8.7, 11.4, 14.0, 15.5, 18.7, 20.2, 21.4, 21.5, 22.8, 26.2, and 28.2. For example, a contemplated crystalline form has a powder X-ray diffraction pattern shown in FIG. 15. In one embodiment, the powder X-ray diffraction pattern of the crystalline form was obtained using Cu Kα radiation.

In another embodiment, disclosed herein is a crystalline form of ((S)-2-amino-3-(3,4-dihydroxyphenyl) propanoyl)-L-tyrosine characterized by a powder X-ray diffraction pattern having a characteristic peak in degrees 2θ at about 8.7 (referred to herein as “Form Q”).

In one embodiment, the crystalline Form Q is characterized by a powder X-ray diffraction pattern that has a characteristic peak in degrees 2θ at about 8.7, is characterized by a powder X-ray diffraction pattern that has a characteristic peak in degrees 2θ at about 13.5, is characterized by a powder X-ray diffraction pattern that has a characteristic peak in degrees 2θ at about 15.4, is characterized by a powder X-ray diffraction pattern that has a characteristic peak in degrees 2θ at about 19.2, is characterized by a powder X-ray diffraction pattern that has a characteristic peak in degrees 2θ at about 20.9, is characterized by a powder X-ray diffraction pattern that has a characteristic peak in degrees 2θ at about 21.6, is characterized by a powder X-ray diffraction pattern that has a characteristic peak in degrees 2θ at about 22.2, is characterized by a powder X-ray diffraction pattern that has a characteristic peak in degrees 2θ at about 25.4, is characterized by a powder X-ray diffraction pattern that has a characteristic peak in degrees 2θ at about 27.8, and/or is characterized by a powder X-ray diffraction pattern that has a characteristic peak in degrees 2θ at about 29.7. In another embodiment, crystalline Form Q is characterized by a powder X-ray diffraction pattern having at least one or more characteristic peaks in degrees 2θ at about 8.7, 13.5, 15.4, 19.2, 20.9, 21.6, 22.2, 25.4, 27.8, and 29.7. For example, a contemplated crystalline form has a powder X-ray diffraction pattern shown in FIG. 15. In one embodiment, the powder X-ray diffraction pattern of the crystalline form was obtained using Cu Kα radiation.

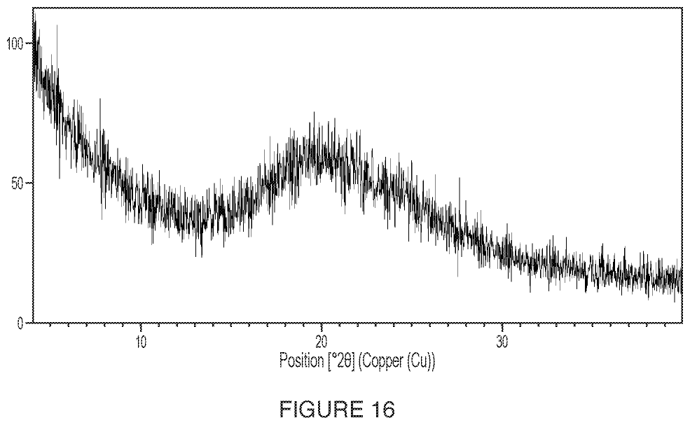

In another embodiment, a substantially amorphous form of ((S)-2-amino-3-(3,4-dihydroxyphenyl) propanoyl)-L-tyrosine is disclosed herein. For example, a contemplated amorphous form has a powder X-ray diffraction pattern shown in FIG. 16.

Embodiments of the disclosure are directed to a highly pure form of ((S)-2-amino-3-(3,4-dihydroxyphenyl) propanoyl)-L-tyrosine. According to some embodiments, ((S)-2-amino-3-(3,4-dihydroxyphenyl) propanoyl)-L-tyrosine has a purity above about 97%, about 97.5%, about 98%, about 98.5%, about 99%, about 99.5%, about 99.6%, about 99.75%, or about 99.9%. In some embodiments, ((S)-2-amino-3-(3,4-dihydroxyphenyl) propanoyl)-L-tyrosine has a purity above about 99%. In some embodiments, ((S)-2-amino-3-(3,4-dihydroxyphenyl) propanoyl)-L-tyrosine has a purity above about 99.5%. In some embodiments, ((S)-2-amino-3-(3,4-dihydroxyphenyl) propanoyl)-L-tyrosine has a purity above about 99.6%. In some embodiments, ((S)-2-amino-3-(3,4-dihydroxyphenyl) propanoyl)-L-tyrosine has a purity above about 99.9%. According to some embodiments, the highly pure form of ((S)-2-amino-3-(3,4-dihydroxyphenyl) propanoyl)-L-tyrosine is polymorph N.

According to some embodiments, the highly pure form of ((S)-2-amino-3-(3,4-dihydroxyphenyl) propanoyl)-L-tyrosine is characterized by an HPLC chromatogram having a peak at about 22.65 RT. In some embodiments, the HPLC chromatogram has peaks at about 16.86, 18.45, 21.79, 22.65, 23.90, 25.99, 26.62, 30.39, 30.83, 31.90, 32.20, 32.76, 33.43, 34.95, 37.01, and 39.92 RT. In some other embodiments, the highly pure form of ((S)-2-amino-3-(3,4-dihydroxyphenyl) propanoyl)-L-tyrosine is characterized by an HPLC chromatogram having a peak at about 22.56 RT. In some embodiments, the HPLC chromatogram has peaks at about 5.81, 20.81, 21.71, 22.56, 33.38, and 36.88 RT. In some embodiments, the HPLC chromatogram has peaks at about 5.80, 20.82, 21.72, 22.56, 33.39, and 36.89 RT. According to some embodiments, the highly pure form of ((S)-2-amino-3-(3,4-dihydroxyphenyl) propanoyl)-L-tyrosine is characterized by an HPLC chromatogram having a peak at about 22.55 RT. In some embodiments, the HPLC chromatogram has peaks at about 5.80, 20.81, 21.70, 22.55, 33.37, and 36.87 RT. According to some embodiments, the highly pure form of ((S)-2-amino-3-(3,4-dihydroxyphenyl) propanoyl)-L-tyrosine is characterized by an HPLC chromatogram having a peak at about 22.54 RT. In some embodiments, the HPLC chromatogram has peaks at about 5.80, 20.80, 21.70, 22.54, 33.38, and 36.88 RT.

According to some embodiments, the color of the ((S)-2-amino-3-(3,4-dihydroxyphenyl) propanoyl)-L-tyrosine, e.g., polymorph N, prepared according to any of the processes detailed herein, is defined as Y5, Y6, Y7, B5, B6, B7, BY5, BY6 or BY7 when dissolved in TRIS buffer. It is noted that the color scales referred to herein are Color Reference Solutions Y1-Y7, relating to Ph. Eur. Y1-Y7 Certipur®, BY1-BY7, relating to Ph. Eur. BY1-BY7 Certipur®, and B1-B7, relating to Ph. Eur. B1-B7 Certipur®.

According to some embodiments, the crystalline form of ((S)-2-amino-3-(3,4-dihydroxyphenyl) propanoyl)-L-tyrosine disclosed herein is purified from an already prepared batch of crystalline Form N of ((S)-2-amino-3-(3,4-dihydroxyphenyl) propanoyl)-L-tyrosine.

((S)-2-amino-3-(3,4-dihydroxyphenyl) propanoyl)-L-tyrosine may be purified to remove impurities causing color. According to some embodiments, active carbon may be used to purify ((S)-2-amino-3-(3,4-dihydroxyphenyl) propanoyl)-L-tyrosine. According to some embodiments, the active carbon is selected from newcarb, pencarb, Alquandracarb, Eno PC Carb, HW Carb, Tri Carb, C-CA Activated carbon, C-HA Activated carbon, C-VA Activated carbon, C-VW Activated carbon, norite carbon, penta carb, or any combination thereof. According to some embodiments, one portion of active carbon may be used, wherein, in this context, a single portion is considered one step of the purification process, wherein the active carbon is added to the vessel to purify the material found therein. According to some embodiments, two, three, four, five or more portions of active carbon may be used. In some embodiments, at least two portions of active carbon are used in the process. According to some embodiments, each portion of active carbon comprises one type of active carbon. According to some embodiments, each portion of active carbon comprises two, three, four, five, or more, types of active carbon. According to some embodiments, if more than one portion of active carbon is used, the type of active carbon used in each portion may be different from one another. According to some embodiments, if more than one portion of active carbon is used, the type of active carbon used in each portion may be the same.

According to some embodiments, the amount of active carbon added in each portion is about 5% w/w to about 50% w/w, compared to the weight of the crystalline compound being purified. According to some embodiments, the amount of active carbon added in each portion is about 5% w/w to about 10% w/w, compared to the weight of the crystalline compound being purified. According to some embodiments, the amount of active carbon added in each portion is about 10% w/w to about 50% w/w, compared to the weight of the crystalline compound being purified. According to some embodiments, the amount of active carbon added in each portion is about 10% w/w to about 15% w/w, compared to the weight of the crystalline compound being purified. According to some embodiments, the amount of active carbon added in each portion is about 15% w/w to about 20% w/w, compared to the weight of the crystalline compound being purified. According to some embodiments, the amount of active carbon added in each portion is about 20% w/w to about 25% w/w, compared to the weight of the crystalline compound being purified. According to some embodiments, the amount of active carbon added in each portion is about 25% w/w to about 30% w/w, compared to the weight of the crystalline compound being purified. According to some embodiments, the amount of active carbon added in each portion is about 30% w/w to about 35% w/w, compared to the weight of the crystalline compound being purified. According to some embodiments, the amount of active carbon added in each portion is about 35% w/w to about 40% w/w, compared to the weight of the crystalline compound being purified. According to some embodiments, the amount of active carbon added in each portion is about 40% w/w to about 45% w/w, compared to the weight of the crystalline compound being purified. According to some embodiments, the amount of active carbon added in each portion is about 45% w/w to about 50% w/w, compared to the weight of the crystalline compound being purified. According to some embodiments, the amount of active carbon added in each portion is about 10% w/w, 15% w/w, 20% w/w, 25% w/w, 30% w/w, 35% w/w, 40% w/w, 45% w/w, or 50% w/w, compared to the weight of the crystalline compound being purified. According to some embodiments, the amount of active carbon added in each portion is about 15% w/w, 20% w/w, 25% w/w, or 30% w/w, compared to the weight of the crystalline compound being purified. According to some embodiments, the amount of active carbon added in each portion is about 15% w/w, compared to the weight of the crystalline compound being purified.

According to some other embodiments, acidic agents may be used to purify ((S)-2-amino-3-(3,4-dihydroxyphenyl) propanoyl)-L-tyrosine. Exemplary acidic agents include, but are not limited to, HF, HBr in e.g., acetic acid, trifluoroacetic acid and thioanisole, and BBr₃, BCl₃, or BF₃ etherates, or a combination thereof. According to some embodiments, one portion of acidic agent may be used, wherein, in this context, a single portion is considered one step of the purification process, wherein the acidic agent is added to the vessel to purify the material found therein. According to some embodiments, two, three, four, five or more portions of acidic agent may be used. According to some embodiments, each portion of acidic agent comprises one type of acidic agent. According to some embodiments, each portion of acidic agent comprises two, three, four, five, or more, types of acidic agent. According to some embodiments, if more than one portion of acidic agent is used, the type of acidic agent used in each portion may be different from one another. According to some embodiments, if more than one portion of acidic agent is used, the type of acidic agent used in each portion may be the same.

According to some embodiments, ((S)-2-amino-3-(3,4-dihydroxyphenyl) propanoyl)-L-tyrosine is first prepared, and then purified, including color purification. According to other embodiments, ((S)-2-amino-3-(3,4-dihydroxyphenyl) propanoyl)-L-tyrosine is prepared from

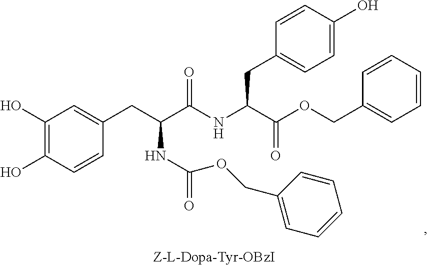 [SMILES: O=C(N[C@@H](Cc1ccc(O)c(O)c1)C(=O)N[C@@H](Cc1ccc(O)cc1)C(=O)OCc1ccccc1)OCc1ccccc1]

wherein the material is purified during the preparation process. For example, the purification process if performed during the preparation of crystalline form N from

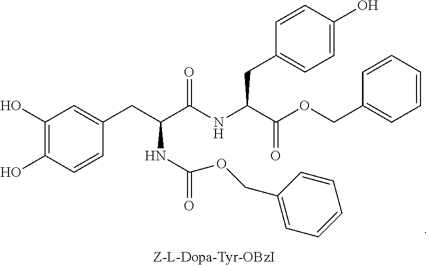 [SMILES: O=C(N[C@@H](Cc1ccc(O)c(O)c1)C(=O)N[C@@H](Cc1ccc(O)cc1)C(=O)OCc1ccccc1)OCc1ccccc1]

According to some embodiments, ((S)-2-amino-3-(3,4-dihydroxyphenyl) propanoyl)-L-tyrosine is purified in an alcohol. According to some embodiments, the alcohol is methanol, ethanol, propanol, iso-propanol, butanol, iso-butanol, or any other appropriate alcohol. According to some embodiments, the alcohol is methanol.

In a further embodiment, a pharmaceutical composition comprising a disclosed crystalline form of ((S)-2-amino-3-(3,4-dihydroxyphenyl) propanoyl)-L-tyrosine and a pharmaceutically acceptable excipient is disclosed herein. For example, a pharmaceutical composition comprising the crystalline Form N of ((S)-2-amino-3-(3,4-dihydroxyphenyl) propanoyl)-L-tyrosine and a pharmaceutically acceptable excipient is disclosed herein. In another embodiment, a pharmaceutical composition formed from a disclosed crystalline form of ((S)-2-amino-3-(3,4-dihydroxyphenyl) propanoyl)-L-tyrosine is disclosed herein. For example, a pharmaceutical composition formed from the crystalline Form N of ((S)-2-amino-3-(3,4-dihydroxyphenyl) propanoyl)-L-tyrosine is disclosed herein.

In yet another embodiment, a pharmaceutical composition comprising a disclosed amorphous form of ((S)-2-amino-3-(3,4-dihydroxyphenyl) propanoyl)-L-tyrosine and a pharmaceutically acceptable excipient is disclosed herein.

In an embodiment, a drug substance comprising at least a detectable amount of a disclosed crystalline form of ((S)-2-amino-3-(3,4-dihydroxyphenyl) propanoyl)-L-tyrosine is disclosed herein. In another embodiment, a drug substance comprising a substantially pure crystalline form of ((S)-2-amino-3-(3,4-dihydroxyphenyl) propanoyl)-L-tyrosine is disclosed herein. For example, a drug substance comprising a substantially pure crystalline Form N of ((S)-2-amino-3-(3,4-dihydroxyphenyl) propanoyl)-L-tyrosine is disclosed herein.

### Compositions

Another aspect of the disclosure provides pharmaceutical compositions comprising crystalline compounds as disclosed herein formulated together with a pharmaceutically acceptable excipient. In particular, the present disclosure provides pharmaceutical compositions comprising crystalline compounds as disclosed herein formulated together with one or more pharmaceutically acceptable excipients. These formulations include those suitable for oral, topical (e.g., transdermal), buccal, ocular, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral, subcutaneous or intravenous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic excipient or excipient suitable for external, enteral or parenteral applications. The active ingredient may be compounded, for example, with the usual non-toxic, pharmaceutically acceptable excipients for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical excipient, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable excipients, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, nano-suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or excipients comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition includes powders, sprays, ointments, pastes, creams, lotions, gels, solutions, patches and inhalants. The active component may be mixed under sterile conditions with a pharmaceutically acceptable excipient, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable excipients and stabilizers. The excipients and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous excipients which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants. For example, crystalline forms provided herein may be milled to obtain a particular particle size, and in at least some embodiments, such crystalline forms may remain substantially stable upon milling.

Amounts of a crystalline compound as described herein in a formulation may vary according to factors such as the disease state, age, sex, and weight of the individual. Dosage regimens can be adjusted to provide the optimum therapeutic response. For example, a single bolus can be administered, several divided doses may be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active crystalline compound calculated to produce the desired therapeutic effect in association with the required pharmaceutical carrier.

The specification for the dosage unit forms of the disclosure are dictated by and directly dependent on (a) the unique characteristics of the crystalline compound selected and the particular therapeutic effect to be achieved, and (b) the limitations inherent in the art of compounding such an active crystalline compound for the treatment of sensitivity in individuals.

Disclosed compositions can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is suitable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin.

A disclosed crystalline compound can be administered in a time release formulation, for example in a composition which includes a slow release polymer. The crystalline compound can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are generally known to those skilled in the art.

In accordance with an alternative aspect of the disclosure, a disclosed crystalline compound can be formulated with one or more additional compounds that enhance the solubility of the compound.

### Methods

In some embodiments, the disclosure provides a method of treating a neurodegenerative condition and/or a condition characterized by reduced levels of dopamine in the brain in a patient in need thereof, comprises administering to the patient an effective amount of a disclosed crystalline compound, for example, a disclosed crystalline form of ((S)-2-amino-3-(3,4-dihydroxyphenyl) propanoyl)-L-tyrosine. In other embodiments, the disclosure provides a method of treating a neurodegenerative condition and/or a condition characterized by reduced levels of dopamine in the brain in a patient in need thereof, comprising administering to the patient an effective amount of a pharmaceutical composition comprising a disclosed crystalline compound, for example, a disclosed crystalline form of ((S)-2-amino-3-(3,4-dihydroxyphenyl) propanoyl)-L-tyrosine, and a pharmaceutically acceptable excipient.

For example, the crystalline forms of ((S)-2-amino-3-(3,4-dihydroxyphenyl) propanoyl)-L-tyrosine of the present disclosure are useful for prevention or treatment of Parkinson's disease, secondary parkinsonism, Huntington's disease, Parkinson's disease-like syndrome, progressive supranuclear palsy (PSP), multiple system atrophy (MSA), amyotrophic lateral sclerosis (ALS), Shy-Drager syndrome, dystonia, Alzheimer's disease, Lewy body dementias (LBD), akinesia, bradykinesia, and hypokinesia. Further, the crystalline forms of ((S)-2-amino-3-(3,4-dihydroxyphenyl) propanoyl)-L-tyrosine of the present disclosure are useful for prevention or treatment of diseases or symptoms caused by a brain damage including carbon monoxide poisoning or manganese poisoning, or diseases or symptoms associated with neurological diseases or neurological disorders including alcoholism, drug addiction or erectile dysfunction.

In some embodiments, the neurodegenerative condition and/or condition characterized by reduced levels of dopamine in the brain is Parkinson's disease. For example, disclosed herein is a method of treating Parkinson's disease in a patient in need thereof, comprises administering to the patient an effective amount of a disclosed crystalline compound, for example, a disclosed crystalline form of ((S)-2-amino-3-(3,4-dihydroxyphenyl) propanoyl)-L-tyrosine. In other embodiments, the disclosure provides a method of treating Parkinson's disease in a patient in need thereof, comprising administering to the patient an effective amount of a pharmaceutical composition comprising a disclosed crystalline compound, for example, a disclosed crystalline form of ((S)-2-amino-3-(3,4-dihydroxyphenyl) propanoyl)-L-tyrosine, and a pharmaceutically acceptable excipient.

In particular, in certain embodiments, the disclosure provides a method of treating the above medical indications comprising administering to a patient in need thereof an effective amount of a crystalline compound disclosed herein. In certain other embodiments, the disclosure provides a method of treating the above medical conditions in a patient in need thereof, comprising orally, subcutaneously, or intravenously administering to the patient a composition comprising a disclosed crystalline form.

The crystalline compounds disclosed herein can be used as a medicament or pharmaceutically acceptable composition, e.g., in the form of pharmaceutical preparations for oral, enteral, parenteral, or topical administration, and the contemplated methods disclosed herein may include administering orally, enterally, parenterally, or topically a disclosed crystalline compound, or a composition comprising or formed from such a disclosed crystalline compound. For example, a disclosed crystalline form may be capable of controlling one or more pharmacokinetic properties (e.g., a longer or shorter release profile) when administered by a certain route (e.g., oral) or in a certain formulation, as compared to a different route (e.g., subcutaneous) or other formulation e.g., a formulation having the amorphous form. In one embodiment, a disclosed crystalline form may afford substantial reproducibility from one formulation to another.

### EXAMPLES

The compounds described herein can be prepared in a number of ways based on the teachings contained herein and synthetic procedures known in the art. The following non-limiting examples illustrate the disclosure.

### Instrumentation

X-ray owder diffraction (XRPD) analyses were performed using a Panalytical Empyrean diffractometer equipped with a Cu X-ray tube and a PIXcel 1D-Medipix3 detector system. The samples were analysed at ambient temperature in transmission mode and held between low density PVC films. The Almac default XRPD program was used (range 4-40° 20, step size 0.01313°, counting time 92 sec, ˜20 min run time, counting time 46 sec, ˜10 min run time, counting time 23 sec, ˜5 min run time). Samples were spun at 60 rpm during data collection. XRPD patterns were sorted and manipulated using HighScore Plus v4.9 software.

Differential scanning calorimetry (DSC) analyses were carried out on a Perkin Elmer Jade Differential Scanning calorimeter or Perkin Elmer DSC8500 Differential Scanning calorimeter. Accurately weighed samples were placed in crimped aluminium pans (i.e. closed but not gas tight). Each sample was heated under nitrogen at a rate of 10° C./minute to a maximum of 300° C. Indium metal was used as the calibration standard.

Thermogravimetric analyses were carried out on a Mettler Toledo TGA/DSC1 STARe. The calibration standards were indium and tin. Samples were placed in an aluminium sample pan, inserted into the TG furnace and accurately weighed. Under a stream of nitrogen at a rate of 10° C./minute, the heat flow signal was stabilised for one minute at 30° C., prior to heating to 300° C.

Thermogravimetric analyses were also carried out on a TA Instruments Discovery TGA 550 instrument. A standard aluminium pan and lid was positioned on a 100 μL platinum pan and tared. Approximately 5-10 mg of material was carefully placed in the aluminium pan and the lid crimped into position. The sample is heated to 300° C. under a nitrogen environment at 10° C./minute.

Dynamic vapour sorption (DVS) was performed using a Hiden Analytical Instruments IGAsorp Vapour Sorption Balance. Approximately 30 mg of sample was placed into a wire-mesh vapour sorption balance pan, loaded into the IGAsorp vapour sorption balance and held at 25° C.±0.1° C. The sample was subjected to a step profile in 10% increments from 40-90% RH, followed by desorption from 90-0% RH and a second sorption cycle form 0-40% RH. The equilibrium criterion was set to 99.0% step completion within a minimum of 60 minutes and a maximum of 5 hours for each increment. The weight change during the sorption cycle was monitored, allowing for the hygroscopic nature of the sample to be determined. The data collection interval was in seconds.

Volumetric Karl Fischer (KF) analysis was performed using a Mettler Toledo V30 KF titrator. A weighed amount of solid sample was added directly to the KF cell. The solution was stirred and the water content of the sample was then determined by automatic titration against standard KF reagent titrant. The procedure was carried out in duplicate and an average water content reported.

Color determination, as referenced in Examples 18a, 18b, 18c, and 19, was performed using Certipur® Color Reference solutions (Series BY→BY1 to BY7; Series Y→Y1 to Y7). Color Reagent Test (CRT) samples were prepared (per 20 mL) by mixing 20 mg of potassium bisulfite and 3.2 gram of Tris base in 16 gr of water until full dissolution (pH=10.5±0.3). For the color reaction, 375 mg of ((S)-2-amino-3-(3,4-dihydroxyphenyl) propanoyl)-L-tyrosine was added 5 mL of CRT solution and stirred until dissolution and filtered through a PES 0.22 μm syringe filter. Within about an hour of the color reaction, the color of the reacted solution was determined. Reference and reaction solutions were placed in diffused daylight or in light box and viewed vertically against a white background. Solutions were assigned to Y3 to Y7 or BY6 to BY7 specification.

### Screening Methods

### Procedure A: Slurry Experiments

Sufficient ((S)-2-amino-3-(3,4-dihydroxyphenyl) propanoyl)-L-tyrosine (amorphous) was added to a given solvent until undissolved solids remained at the desired temperature (5, 20 or 50° C.). The vial was sealed and the slurry was maintained at the selected temperature and agitated by magnetic stirring for 7 days. Solids were isolated by filtration/centrifugation and air-dried for 1-2 hours prior to analysis by XRPD.

### Procedure B: Temperature Cycling

About 50 volumes of the indicated solvent was added to ((S)-2-amino-3-(3,4-dihydroxyphenyl) propanoyl)-L-tyrosine (Form G), such that complete dissolution did not occur. The sample was stirred at 600 rpm and heated from 20° C. to within 3° C. of the solvent's boiling point (or 100° C., whichever was lower) at 0.5° C./minute, then cooled to 20° C. at 0.2° C./minute, and the cycle repeated an additional time. The sample was held at ambient temperature for several hours to effect precipitation, and the recovered solids were analysed by XRPD.

### Procedure C: Vapour Stress

Approximately 25 mg of amorphous ((S)-2-amino-3-(3,4-dihydroxyphenyl) propanoyl)-L-tyrosine was added to a vial and placed unsealed inside a larger sealed vessel containing the indicated solvent. After ˜7 days, the sample was removed and analysed by XRPD.

### Procedure D: Planetary Milling

Approximately 20 mg of ((S)-2-amino-3-(3,4-dihydroxyphenyl) propanoyl)-L-tyrosine (Form G) was added to a vial with steel milling balls and solvent (10 μL). The vial was sealed and its contents milled using a planetary mill for 30 minutes at a rotation speed of 400 rpm, with 30 minutes pause between milling cycles (24 cycles). The resultant milled material was analysed using XRPD.

### Example 1

Crystalline, Form N material of ((S)-2-amino-3-(3,4-dihydroxyphenyl) propanoyl)-L-tyrosine according to Procedure A at 20° C. using acetone/water (18:82% v/v) as solvent. The XRPD pattern of crystalline Form N is shown in FIG. 1. Characteristic peaks include one or more of the peaks shown in Table 1.

TABLE 1

| Pos. [°2θ] | d-spacing [Å] | Height [cts] | Rel. Int. [%] |
|---|---|---|---|
| 7.6942 | 11.49032 | 545.38 | 28.81 |
| 8.6561 | 10.21558 | 477.88 | 25.25 |
| 8.776 | 10.07628 | 201.23 | 10.63 |
| 12.6099 | 7.01998 | 659.34 | 34.83 |
| 14.3273 | 6.18214 | 41.74 | 2.21 |
| 15.4072 | 5.75116 | 60.97 | 3.22 |
| 16.2143 | 5.46667 | 264.24 | 13.96 |
| 16.3466 | 5.42274 | 1055.1 | 55.74 |
| 16.6697 | 5.31835 | 78.51 | 4.15 |
| 17.3325 | 5.11643 | 280.3 | 14.81 |
| 17.5848 | 5.04358 | 68.63 | 3.63 |
| 20.0874 | 4.42053 | 1892.94 | 100 |
| 20.9763 | 4.23517 | 82.81 | 4.37 |
| 21.8055 | 4.07596 | 94.04 | 4.97 |
| 22.7727 | 3.90498 | 184.78 | 9.76 |
| 23.0824 | 3.85329 | 85.01 | 4.49 |
| 24.1173 | 3.69023 | 87.5 | 4.62 |
| 25.6911 | 3.46763 | 597.47 | 31.56 |
| 26.1292 | 3.41048 | 45.15 | 2.39 |
| 26.5305 | 3.3598 | 72.06 | 3.81 |
| 27.2215 | 3.27606 | 157.01 | 8.29 |
| 27.466 | 3.24745 | 98.22 | 5.19 |
| 27.7698 | 3.21261 | 81.87 | 4.32 |
| 28.0388 | 3.1824 | 56.62 | 2.99 |
| 28.3309 | 3.15024 | 55.76 | 2.95 |
| 28.7397 | 3.10379 | 58.74 | 3.1 |
| 28.8736 | 3.09226 | 112.54 | 5.95 |
| 29.8426 | 2.99402 | 207.88 | 10.98 |
| 30.0041 | 2.97827 | 397.31 | 20.99 |
| 31.0423 | 2.88099 | 10.27 | 0.54 |
| 32.1643 | 2.78301 | 60.08 | 3.17 |
| 32.7004 | 2.73633 | 15.27 | 0.81 |
| 33.0233 | 2.71256 | 34.95 | 1.85 |
| 33.6893 | 2.66044 | 69.17 | 3.65 |
| 34.0754 | 2.629 | 37.22 | 1.97 |
| 34.2426 | 2.61871 | 199 | 10.51 |
| 34.5108 | 2.59682 | 27.5 | 1.45 |
| 35.2134 | 2.54871 | 46.46 | 2.45 |
| 36.1035 | 2.48789 | 70.52 | 3.73 |
| 37.4082 | 2.40406 | 41.42 | 2.19 |
| 38.3966 | 2.34442 | 6.71 | 0.35 |
| 39.1336 | 2.30195 | 23.1 | 1.22 |

FIG. 2 depicts the differential scanning calorimetry (DSC) profile of crystalline Form N. As shown in FIG. 2, crystalline Form N shows a characteristic broad endotherm with an onset of about 135° C. and a peak of about 159° C.; a characteristic endotherm with an onset of about 174° C. and a peak of about 176° C.; a characteristic exotherm with an onset of about 177° C. and a peak of about 178° C.; and a characteristic endotherm with an onset of about 272° C. and a peak of about 278° C.

Crystalline Form N of ((S)-2-amino-3-(3,4-dihydroxyphenyl) propanoyl)-L-tyrosine displayed a thermogravimetric analysis (TGA) profile showing a mass loss of about 15 wt. % between about 22° C. to about 196° C. (FIG. 3). Crystalline Form N displayed a dynamic vapor sorption (DVS) profile showing a reversable total mass change of about 0.6 wt. % between about 40 to about 80% relative humidity (RH) at 25° C. Karl Fischer (KF) analysis showed 15.6% water by weight (about 3.7 equivalent by molar ratio).

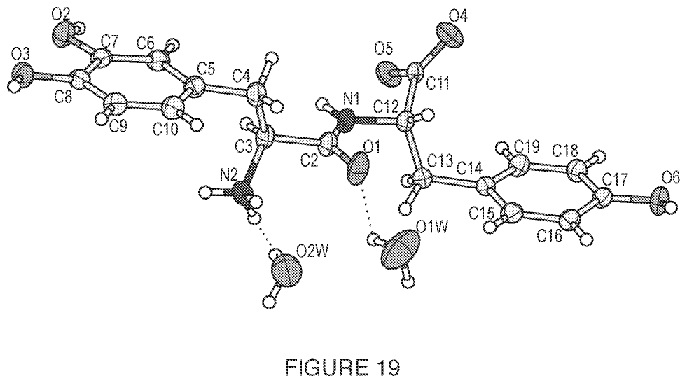

Single crystals of Form N were grown by vapor diffusion using water:DMSO (9:5 v/v) as solvent and trifluoroethanol as anti-solvent, and analyzed by single crystal X-ray analysis. The crystal was observed to be a dihydrate and displayed a monoclinic system with the unit cell parameters, a C2 space group, and other crystallographic details shown in Table 1A, below. The X-ray crystal structure is shown in FIG. 19, with hydrogen atoms omitted for clarity.

TABLE 1A

| Empirical formula | C₁₈H₂₄N₂O₈ |
|---|---|
| Formula weight | 396.39 |
| Temperature/K | 293 |
| Crystal system | monoclinic |
| Space group | C2 |
| a/Å | 25.364(2) |
| b/Å | 7.3295(6) |
| c/Å | 11.2857(9) |
| α/° | 90 |
| β/° | 115.570(2) |
| γ/° | 90 |
| Volume/Å³ | 1892.6(3) |
| Z | 4 |
| ρcalcg/cm³ | 1.391 |
| μ/mm−1 | 0.932 |
| F(000) | 840.0 |
| Crystal size/mm³ | 0.36 × 0.34 × 0.18 |
| Radiation | CuKα (λ = 1.54178) |
| 2Θ range for data collection/° | 7.728 to 149.104 |
| Index ranges | −30 ≤ h ≤ 31, −9 ≤ k ≤ 9, −13 ≤ |
| | l ≤ 14 |
| Reflections collected | 24590 |
| Independent reflections | 3820 [Rint = 0.0474, Rsigma = 0.0368] |
| Data/restraints/parameters | 3820/2/263 |
| Goodness-of-fit on F2 | 1.078 |
| Final R indexes [I >= 2σ (I)] | R1 = 0.0480, wR2 = 0.1310 |
| Final R indexes [all data] | R1 = 0.0494, wR2 = 0.1315 |
| Largest diff. peak/hole/e Å−3 | 0.34/−0.25 |
| Flack parameter | 0.2(2) |

### Example 2

The XRPD pattern of crystalline Form A of ((S)-2-amino-3-(3,4-dihydroxyphenyl) propanoyl)-L-tyrosine is shown in FIG. 4. Characteristic peaks include one or more of the peaks shown in Table 2.

TABLE 2

| Pos. [°2θ] | d-spacing [Å] | Height [cts] | Rel. Int. [%] |
|---|---|---|---|
| 4.7464 | 18.60268 | 68.42 | 51.14 |
| 9.4913 | 9.31072 | 72.75 | 54.37 |
| 18.3292 | 4.8364 | 96.2 | 71.9 |
| 18.9812 | 4.67172 | 133.79 | 100 |
| 21.125 | 4.20222 | 45.28 | 33.84 |

Crystalline Form A of ((S)-2-amino-3-(3,4-dihydroxyphenyl) propanoyl)-L-tyrosine displayed a thermogravimetric analysis (TGA) profile showing a mass loss of about 10.2 wt. % between about 40° C. to about 200° C.

### Example 3

Crystalline, Form B material of ((S)-2-amino-3-(3,4-dihydroxyphenyl) propanoyl)-L-tyrosine was prepared by recrystallization of Form G from water. Form B was also prepared according to Procedure A at 5° C. using dioxane/water (82:18% v/v) as solvent. The XRPD pattern of crystalline Form B is shown in FIG. 4. Characteristic peaks include one or more of the peaks shown in Table 3.

TABLE 3

| Pos. [°2θ] | d-spacing [Å] | Height [cts] | Rel. Int. [%] |
|---|---|---|---|
| 7.2727 | 12.15533 | 113.57 | 12.44 |
| 9.9434 | 8.8957 | 128.61 | 14.09 |
| 10.6504 | 8.29991 | 37.06 | 4.06 |
| 12.3142 | 7.18787 | 28.56 | 3.13 |
| 14.4781 | 6.11809 | 458.56 | 50.23 |
| 14.612 | 6.06231 | 690.49 | 75.64 |
| 14.8065 | 5.98311 | 298.88 | 32.74 |
| 15.5236 | 5.70831 | 80.63 | 8.83 |
| 16.3008 | 5.43336 | 37.46 | 4.1 |
| 17.7203 | 5.00532 | 379.77 | 41.6 |
| 19.9726 | 4.44568 | 112.47 | 12.32 |
| 20.3171 | 4.37107 | 153.29 | 16.79 |
| 21.3019 | 4.17116 | 104.92 | 11.49 |
| 22.0384 | 4.0334 | 32.94 | 3.61 |
| 22.8821 | 3.88657 | 634.96 | 69.56 |
| 23.1703 | 3.83886 | 912.88 | 100 |
| 24.0298 | 3.70348 | 139.27 | 15.26 |
| 24.5229 | 3.6271 | 46.87 | 5.13 |
| 25.433 | 3.50223 | 564.17 | 61.8 |
| 26.0148 | 3.42522 | 83.72 | 9.17 |
| 26.4072 | 3.37521 | 63.91 | 7 |
| 27.27 | 3.27034 | 145.35 | 15.92 |
| 27.6742 | 3.22083 | 36.11 | 3.96 |
| 28.6352 | 3.11746 | 63.91 | 7 |
| 29.4678 | 3.03124 | 227.96 | 24.97 |
| 30.163 | 2.96295 | 219.39 | 24.03 |
| 31.3209 | 2.856 | 174.13 | 19.07 |
| 32.008 | 2.79624 | 38.45 | 4.21 |
| 32.6353 | 2.74391 | 62.31 | 6.83 |
| 33.2674 | 2.69321 | 98.82 | 10.83 |
| 34.8018 | 2.57791 | 40.4 | 4.43 |
| 35.5774 | 2.52346 | 28.64 | 3.14 |
| 36.3063 | 2.47446 | 21.54 | 2.36 |
| 37.0266 | 2.42796 | 74.06 | 8.11 |
| 38.1635 | 2.3582 | 21.17 | 2.32 |
| 39.1651 | 2.30017 | 33.68 | 3.69 |

FIG. 5 depicts the differential scanning calorimetry (DSC) profile of crystalline Form B. As shown in FIG. 5, crystalline Form B shows a characteristic endotherm with an onset of about 96° C. and a peak of about 119° C.; a characteristic endotherm with an onset of about 161° C. and a peak of about 176° C.; and a characteristic endotherm with an onset of about 275° C. and a peak of about 280° C. Crystalline Form B displayed a thermogravimetric analysis (TGA) profile showing a mass loss of about 15.6 wt. % between about 40° C. to about 200° C. Karl Fischer (KF) analysis showed 17.2% water by weight (about 2.2 equivalent by molar ratio).

### Example 4

Crystalline, Form C material of ((S)-2-amino-3-(3,4-dihydroxyphenyl) propanoyl)-L-tyrosine was according to Procedure A at 5° C. and 50° C. using ethanol/water (96:4% v/v) as solvent. Crystalline Form C was also prepared according to Procedure C using ethanol/water (96:4% v/v) as solvent. The XRPD pattern of crystalline Form C is shown in FIG. 4. Characteristic peaks include one or more of the peaks shown in Table 4.

TABLE 4

| Pos. [°2θ] | d-spacing [Å] | Height [cts] | Rel. Int. [%] |
|---|---|---|---|
| 7.6993 | 11.4828 | 260.63 | 17.49 |
| 8.9562 | 9.87394 | 1490.22 | 100 |
| 10.9081 | 8.11105 | 42.54 | 2.85 |
| 13.3441 | 6.63533 | 104.38 | 7 |
| 13.5378 | 6.54087 | 140.63 | 9.44 |
| 15.223 | 5.82036 | 108.42 | 7.28 |
| 15.4494 | 5.73558 | 557.97 | 37.44 |
| 19.6712 | 4.51311 | 82.34 | 5.53 |
| 19.9776 | 4.44459 | 79.33 | 5.32 |
| 20.2041 | 4.39162 | 37.07 | 2.49 |
| 20.5108 | 4.33023 | 657.49 | 44.12 |
| 21.2456 | 4.18209 | 71.79 | 4.82 |
| 21.7064 | 4.09095 | 37.43 | 2.51 |
| 22.089 | 4.02429 | 566.52 | 38.02 |
| 22.4338 | 3.9632 | 52.35 | 3.51 |
| 22.8097 | 3.89873 | 36.59 | 2.46 |
| 23.2387 | 3.82455 | 30.93 | 2.08 |
| 24.6737 | 3.60826 | 53.03 | 3.56 |
| 25.7041 | 3.4659 | 258.74 | 17.36 |
| 26.6497 | 3.34504 | 41.61 | 2.79 |
| 27.1082 | 3.2895 | 29.87 | 2 |
| 27.897 | 3.19825 | 163.31 | 10.96 |
| 28.1074 | 3.17479 | 120.13 | 8.06 |
| 28.929 | 3.08391 | 27.9 | 1.87 |
| 29.1213 | 3.06652 | 53.37 | 3.58 |
| 29.4623 | 3.02929 | 33.59 | 2.25 |
| 29.8379 | 2.99201 | 39.13 | 2.63 |
| 30.0482 | 2.97154 | 50.62 | 3.4 |

FIG. 6 depicts the differential scanning calorimetry (DSC) profile of crystalline Form C. As shown in FIG. 6, crystalline Form C shows a characteristic endotherm with an onset of about 145° C. and a peak of about 158° C.; a characteristic exotherm with an onset of about 175° C. and a peak of about 178° C.; and a characteristic endotherm with an onset of about 269° C. and a peak of about 275° C. Crystalline Form C displayed a thermogravimetric analysis (TGA) profile showing a mass loss of about 1.75 wt. % between about 30° C. to about 190° C. Crystalline Form C converted to Form G upon storage at ambient conditions.

### Example 5

Crystalline, Form D material of ((S)-2-amino-3-(3,4-dihydroxyphenyl) propanoyl)-L-tyrosine was according to Procedure A at 5° C. or Procedure C, each using water as solvent. The XRPD pattern of crystalline Form D is shown in FIG. 7. Characteristic peaks include one or more of the peaks shown in Table 5.

TABLE 5

| Pos. [°2θ] | d-spacing [Å] | Height [cts] | Rel. Int. [%] |
|---|---|---|---|
| 10.399 | 8.49993 | 51.35 | 8.67 |
| 10.715 | 8.24996 | 59.29 | 10.01 |
| 11.7327 | 7.54277 | 101.57 | 17.16 |
| 12.2303 | 7.23701 | 91.16 | 15.4 |
| 13.243 | 6.68026 | 45.61 | 7.7 |
| 14.266 | 6.20857 | 42.03 | 7.1 |
| 16.1498 | 5.48836 | 66.06 | 11.16 |
| 16.9608 | 5.22771 | 241.12 | 40.73 |
| 17.8037 | 4.98208 | 290.38 | 49.05 |
| 18.2408 | 4.86366 | 79.56 | 13.44 |
| 18.5988 | 4.77084 | 103.83 | 17.54 |
| 18.9058 | 4.69405 | 592.01 | 100 |
| 19.1669 | 4.6307 | 334.36 | 56.48 |
| 19.5218 | 4.54731 | 90.72 | 15.32 |
| 20.8697 | 4.25657 | 174.04 | 29.4 |
| 21.1163 | 4.20741 | 87.68 | 14.81 |
| 21.8135 | 4.07449 | 91.18 | 15.4 |
| 22.0304 | 4.03484 | 68.91 | 11.64 |
| 23.2247 | 3.82684 | 45.67 | 7.71 |
| 23.5973 | 3.77036 | 204.99 | 34.63 |
| 23.9378 | 3.7175 | 46.47 | 7.85 |
| 25.0648 | 3.54989 | 35.02 | 5.91 |
| 25.5061 | 3.49237 | 80.82 | 13.65 |
| 26.0984 | 3.41444 | 93.39 | 15.77 |
| 26.6873 | 3.34041 | 177.88 | 30.05 |
| 27.1161 | 3.28855 | 68.7 | 11.6 |
| 27.3272 | 3.26363 | 97.76 | 16.51 |
| 27.8936 | 3.19864 | 37.54 | 6.34 |
| 28.5354 | 3.12814 | 104.75 | 17.69 |
| 28.8717 | 3.09246 | 39.22 | 6.62 |
| 29.6228 | 3.01574 | 77.61 | 13.11 |
| 30.319 | 2.94805 | 58.96 | 9.96 |
| 30.5816 | 2.92334 | 66.2 | 11.18 |
| 31.2444 | 2.86282 | 57.48 | 9.71 |
| 32.9887 | 2.71532 | 26.39 | 4.46 |
| 33.5913 | 2.66798 | 56.55 | 9.55 |
| 34.0852 | 2.63044 | 51.08 | 8.63 |
| 34.3361 | 2.60963 | 35.53 | 6 |
| 34.6521 | 2.58655 | 33.93 | 5.73 |
| 36.1396 | 2.48549 | 36.27 | 6.13 |
| 37.1102 | 2.42268 | 31.94 | 5.4 |
| 38.0012 | 2.3679 | 21.99 | 3.71 |
| 38.4069 | 2.34188 | 27.45 | 4.64 |
| 39.1876 | 2.297 | 24.98 | 4.22 |

FIG. 8 depicts the differential scanning calorimetry (DSC) profile of crystalline Form D. As shown in FIG. 8, crystalline Form D shows a characteristic broad endotherm with an onset of about 79° C. and a peak of about 97° C.; a characteristic endotherm with an onset of about 147° C. and a peak of about 152° C.; a characteristic endotherm with an onset of about 175° C. and a peak of about 176° C.; a characteristic exotherm with an onset of about 178° C.; and a peak of about 179° C.; a characteristic exotherm with an onset of about 183° C. and a peak of about 191° C.; and a characteristic endotherm with an onset of about 263° C. and a peak of about 270° C. Crystalline Form D displayed a thermogravimetric analysis (TGA) profile showing a mass loss of about 38 wt. % between about 30° C. to about 211° C. (FIG. 8). Crystalline Form D converted to Form N upon storage at ambient conditions.

### Example 6

Crystalline, Form E material of ((S)-2-amino-3-(3,4-dihydroxyphenyl) propanoyl)-L-tyrosine was prepared by heating a sample of Form B (˜5 mg) on the TG/DTA instrument to just above the desolvation temperature, and then held at that temperature until weight loss had stabilised (15-20 min). The sample was then cooled to ambient temperature and analysed immediately by XRPD. The XRPD pattern of crystalline Form E is shown in FIG. 7. Characteristic peaks include one or more of the peaks shown in Table 6.

TABLE 6

| Pos. [°2θ] | d-spacing [Å] | Height [cts] | Rel. Int. [%] |
|---|---|---|---|
| 10.107 | 8.74482 | 36.12 | 3.21 |
| 10.7603 | 8.22218 | 145.04 | 12.88 |
| 12.5287 | 7.06531 | 205.53 | 18.24 |
| 14.4095 | 6.14197 | 40.01 | 3.55 |
| 14.7563 | 6.00336 | 1126.48 | 100 |
| 15.0004 | 5.90134 | 51.74 | 4.59 |
| 15.181 | 5.83635 | 111.38 | 9.89 |
| 15.7872 | 5.61358 | 54.21 | 4.81 |
| 16.4938 | 5.37022 | 41.7 | 3.7 |
| 16.9548 | 5.22523 | 32.33 | 2.87 |
| 17.9338 | 4.94621 | 524.98 | 46.6 |
| 18.9027 | 4.69094 | 35.4 | 3.14 |
| 19.1884 | 4.62174 | 30.82 | 2.74 |
| 20.3423 | 4.36572 | 200.07 | 17.76 |
| 20.6711 | 4.29345 | 44.09 | 3.91 |
| 20.9048 | 4.24597 | 62.24 | 5.53 |
| 21.0931 | 4.20849 | 53.88 | 4.78 |
| 22.2164 | 3.99818 | 43.34 | 3.85 |
| 23.2565 | 3.82484 | 217.56 | 19.31 |
| 23.5452 | 3.77859 | 657.56 | 58.37 |
| 24.4331 | 3.64325 | 526.86 | 46.77 |
| 25.8137 | 3.45144 | 278.55 | 24.73 |
| 26.3307 | 3.38483 | 104 | 9.23 |
| 26.7792 | 3.32916 | 48.77 | 4.33 |

Crystalline Form E displayed a thermogravimetric analysis (TGA) profile showing a mass loss of about 11.5 wt. % between about 40° C. to about 200° C. Crystalline Form E displayed a dynamic vapor sorption (DVS) profile showing a reversable total mass change of about 8 wt. % between about 0 to about 90% relative humidity (RH) at 25° C. Crystalline Form E converted to Form B upon storage at ambient conditions.

### Example 7

The XRPD pattern of crystalline Form G of ((S)-2-amino-3-(3,4-dihydroxyphenyl) propanoyl)-L-tyrosine is shown in FIG. 7. Characteristic peaks include one or more of the peaks shown in Table 7.

TABLE 7

| Pos. [°2θ] | d-spacing [Å] | Height [cts] | Rel. Int. [%] |
|---|---|---|---|
| 7.6463 | 11.56222 | 256.96 | 11.73 |
| 8.8063 | 10.04167 | 2191.04 | 100 |
| 9.8727 | 8.95931 | 33.61 | 1.53 |
| 11.1317 | 7.94863 | 114.99 | 5.25 |
| 13.2089 | 6.70295 | 46.68 | 2.13 |
| 13.5002 | 6.55897 | 342.74 | 15.64 |
| 14.8764 | 5.95516 | 42.39 | 1.93 |
| 15.3017 | 5.79061 | 148.23 | 6.77 |
| 15.5243 | 5.70808 | 1103.73 | 50.37 |
| 16.1673 | 5.48246 | 39.35 | 1.8 |
| 17.1044 | 5.18416 | 31.48 | 1.44 |
| 19.4328 | 4.56794 | 277.89 | 12.68 |
| 19.9383 | 4.45325 | 53.45 | 2.44 |
| 20.9524 | 4.23995 | 676.45 | 30.87 |
| 21.4235 | 4.14776 | 67.48 | 3.08 |
| 21.8179 | 4.07367 | 677.54 | 30.92 |
| 22.3282 | 3.9817 | 214.8 | 9.8 |
| 22.7674 | 3.90588 | 48.59 | 2.22 |
| 23.0397 | 3.86033 | 49.71 | 2.27 |
| 24.856 | 3.58221 | 76.68 | 3.5 |
| 25.4353 | 3.50192 | 166 | 7.58 |
| 26.0643 | 3.41883 | 134.67 | 6.15 |
| 26.5984 | 3.35137 | 88.04 | 4.02 |
| 27.8174 | 3.20722 | 181.2 | 8.27 |
| 28.9534 | 3.08392 | 106.13 | 4.84 |
| 29.9306 | 2.98542 | 204.28 | 9.32 |
| 30.6871 | 2.91352 | 45.78 | 2.09 |
| 30.8949 | 2.89201 | 19.6 | 0.89 |
| 31.5749 | 2.83361 | 23.64 | 1.08 |
| 31.9287 | 2.80301 | 20.85 | 0.95 |
| 32.5927 | 2.7474 | 54.5 | 2.49 |
| 33.2049 | 2.69814 | 16.68 | 0.76 |
| 33.7583 | 2.65296 | 25.12 | 1.15 |
| 34.0765 | 2.62892 | 41.83 | 1.91 |
| 34.4702 | 2.60194 | 34.06 | 1.55 |
| 35.7874 | 2.50913 | 42.78 | 1.95 |
| 36.0916 | 2.48663 | 20.96 | 0.96 |

FIG. 9 depicts the differential scanning calorimetry (DSC) profile of crystalline Form G. As shown in FIG. 9, crystalline Form G shows a characteristic endotherm with an onset of about 126° C. and a peak of about 130° C.; a characteristic endotherm with an onset of about 159° C. and a peak of about 169° C.; a characteristic exotherm with an onset of about 171° C.; and a peak of about 176° C.; and a characteristic endotherm with an onset of about 264° C. and a peak of about 271° C.

Crystalline Form G displayed a thermogravimetric analysis (TGA) profile showing a mass loss of about 15 wt. % between about 42° C. to about 208° C. (FIG. 14). Crystalline Form G displayed a dynamic vapor sorption (DVS) profile showing a reversable total mass change of about 2.6 wt. % between about 40 to about 80% relative humidity (RH) at 25° C. Karl Fischer (KF) analysis showed 12.3% water by weight (about 2.3 equivalent by molar ratio).

Crystalline Form G displayed a crystal particle size of <100 μm by polarized light microscopy with significant agglomeration/aggregation, whereas fine crystalline particles <20 μm) were observed with Form N. Crystalline Form N material was less hygroscopic (0.6% w/w gained between 40-80% RH) than Form G material (2.6% w/w between 40-80% RH) and DVS analysis indicated Pattern N material was a more stable hydrate losing significantly less water at low RH.

### Example 8

Crystalline, Form H material of ((S)-2-amino-3-(3,4-dihydroxyphenyl) propanoyl)-L-tyrosine was prepared by heating a sample of Form G (˜5 mg) on the TG/DTA instrument to just above the desolvation temperature, and then held at that temperature until weight loss had stabilised (15-20 min). The sample was then cooled to ambient temperature and analysed immediately by XRPD. The XRPD pattern of crystalline Form H is shown in FIG. 10. Characteristic peaks include one or more of the peaks shown in Table 8.

TABLE 8

| Pos. [°2θ] | d-spacing [Å] | Height [cts] | Rel. Int. [%] |
|---|---|---|---|
| 7.9965 | 11.05661 | 85.02 | 15.91 |
| 8.7361 | 10.12221 | 534.54 | 100 |
| 11.3804 | 7.77552 | 38.2 | 7.15 |
| 12.2886 | 7.19683 | 16.45 | 3.08 |
| 14.1628 | 6.25359 | 30.68 | 5.74 |
| 14.5531 | 6.08673 | 24.17 | 4.52 |
| 15.4593 | 5.72719 | 21.82 | 4.08 |
| 16.0509 | 5.52195 | 108.97 | 20.39 |
| 18.7467 | 4.73353 | 46.74 | 8.74 |
| 21.1563 | 4.19954 | 87.48 | 16.37 |
| 21.3775 | 4.15659 | 230.31 | 43.08 |
| 22.8733 | 3.88804 | 26.63 | 4.98 |
| 24.7634 | 3.5954 | 11.54 | 2.16 |

### Example 9

The XRPD pattern of crystalline Form I of ((S)-2-amino-3-(3,4-dihydroxyphenyl) propanoyl)-L-tyrosine is shown in FIG. 10. Characteristic peaks include one or more of the peaks shown in Table 9. Crystalline Form I displayed a thermogravimetric analysis (TGA) profile showing a mass loss of about 11 wt. % between about 30° C. to about 212° C.

TABLE 9

| Pos. [°2θ] | d-spacing [Å] | Height [cts] | Rel. Int. [%] |
|---|---|---|---|
| 4.3863 | 20.14565 | 1565.21 | 5.49 |
| 7.3949 | 11.95475 | 10139 | 35.57 |
| 7.4747 | 11.8273 | 6312.64 | 22.14 |
| 7.9358 | 11.14112 | 429.56 | 1.51 |
| 8.6839 | 10.18294 | 28507.75 | 100 |
| 8.7822 | 10.0692 | 25810.49 | 90.54 |
| 10.4284 | 8.48304 | 785.26 | 2.75 |
| 10.947 | 8.08231 | 1067.47 | 3.74 |
| 11.3248 | 7.81351 | 539.88 | 1.89 |
| 11.5007 | 7.69445 | 216.66 | 0.76 |
| 11.859 | 7.46275 | 745.9 | 2.62 |
| 12.5282 | 7.06559 | 678.94 | 2.38 |
| 13.1921 | 6.71144 | 71.64 | 0.25 |
| 13.508 | 6.55523 | 170.64 | 0.6 |
| 13.9188 | 6.36263 | 3488.46 | 12.24 |
| 14.2384 | 6.22053 | 3315.42 | 11.63 |
| 14.6051 | 6.06517 | 202.64 | 0.71 |
| 14.7975 | 5.98674 | 549.63 | 1.93 |
| 15.5049 | 5.71515 | 12362.53 | 43.37 |
| 16.357 | 5.41929 | 38.82 | 0.14 |
| 16.9133 | 5.24229 | 138.92 | 0.49 |
| 17.4217 | 5.09043 | 192.66 | 0.68 |
| 17.8097 | 4.98042 | 682.53 | 2.39 |
| 18.2723 | 4.85536 | 1269.79 | 4.45 |
| 18.3729 | 4.82899 | 1311.9 | 4.6 |
| 18.8307 | 4.71261 | 1681.35 | 5.9 |
| 19.4657 | 4.56028 | 231 | 0.81 |
| 20.3989 | 4.35373 | 212.41 | 0.75 |
| 20.6457 | 4.30223 | 127.51 | 0.45 |
| 20.9572 | 4.239 | 436.51 | 1.53 |
| 21.1532 | 4.20016 | 3288.71 | 11.54 |
| 21.5549 | 4.12278 | 7376.04 | 25.87 |
| 21.8528 | 4.06724 | 3784.6 | 13.28 |
| 22.3296 | 3.98146 | 86.09 | 0.3 |
| 22.7444 | 3.90978 | 2383.4 | 8.36 |
| 23.094 | 3.85138 | 1498.48 | 5.26 |
| 23.5712 | 3.77448 | 333.22 | 1.17 |
| 23.8199 | 3.73563 | 794.14 | 2.79 |
| 24.6352 | 3.61381 | 615.37 | 2.16 |
| 24.9239 | 3.5726 | 665.22 | 2.33 |
| 25.3021 | 3.52006 | 85.23 | 0.3 |
| 26.2286 | 3.39778 | 1925.42 | 6.75 |
| 26.4859 | 3.36535 | 978.51 | 3.43 |
| 26.7141 | 3.33712 | 530.35 | 1.86 |
| 28.0558 | 3.18051 | 575.82 | 2.02 |
| 28.2824 | 3.15554 | 872.47 | 3.06 |
| 28.5076 | 3.13112 | 580.11 | 2.03 |
| 29.0126 | 3.07776 | 1102.64 | 3.87 |
| 29.5726 | 3.02074 | 961.74 | 3.37 |
| 29.846 | 2.99369 | 612.46 | 2.15 |
| 31.0057 | 2.88431 | 236.46 | 0.83 |
| 31.403 | 2.84872 | 384.31 | 1.35 |
| 32.229 | 2.77757 | 47.86 | 0.17 |
| 32.6625 | 2.74169 | 94.36 | 0.33 |
| 33.2571 | 2.69402 | 165.41 | 0.58 |
| 33.6971 | 2.65984 | 136 | 0.48 |
| 34.0933 | 2.62983 | 80.84 | 0.28 |
| 34.4779 | 2.60137 | 96.62 | 0.34 |
| 34.9985 | 2.56386 | 116.71 | 0.41 |
| 35.5329 | 2.52652 | 99.47 | 0.35 |
| 36.1334 | 2.48384 | 857 | 3.01 |
| 36.3223 | 2.4734 | 617.47 | 2.17 |
| 36.9468 | 2.43302 | 115.22 | 0.4 |
| 37.9201 | 2.37278 | 96.76 | 0.34 |
| 38.8509 | 2.31805 | 192.06 | 0.67 |

### Example 10

Crystalline, Form J material of ((S)-2-amino-3-(3,4-dihydroxyphenyl) propanoyl)-L-tyrosine was prepared by The XRPD pattern of crystalline Form J is shown in FIG. 10. Characteristic peaks include one or more of the peaks shown in Table 10.

TABLE 10

| Pos. [°2θ] | d-spacing [Å] | Height [cts] | Rel. Int. [%] |
|---|---|---|---|
| 4.3902 | 20.12788 | 262.38 | 7.04 |
| 7.4717 | 11.83197 | 900.12 | 24.14 |
| 7.9204 | 11.16266 | 67.73 | 1.82 |
| 8.7781 | 10.07385 | 3728.05 | 100 |
| 10.4225 | 8.48785 | 100.35 | 2.69 |
| 11.3147 | 7.82048 | 93.71 | 2.51 |
| 12.5463 | 7.05541 | 98.13 | 2.63 |
| 13.4993 | 6.55944 | 40.17 | 1.08 |
| 13.9058 | 6.36859 | 486.57 | 13.05 |
| 14.7849 | 5.99182 | 75.79 | 2.03 |
| 15.4968 | 5.71813 | 1415.05 | 37.96 |
| 16.7715 | 5.28628 | 30.21 | 0.81 |
| 17.7779 | 4.98924 | 103.94 | 2.79 |
| 18.8519 | 4.70736 | 243.3 | 6.53 |
| 19.438 | 4.56672 | 30.03 | 0.81 |
| 20.3449 | 4.36516 | 28.35 | 0.76 |
| 20.9451 | 4.24141 | 106 | 2.84 |
| 21.4198 | 4.14847 | 764.84 | 20.52 |
| 21.5554 | 4.12268 | 779.48 | 20.91 |
| 21.8455 | 4.06858 | 107.79 | 2.89 |
| 22.713 | 3.91512 | 294.41 | 7.9 |
| 23.1914 | 3.83542 | 49.74 | 1.33 |
| 24.6526 | 3.61131 | 85.8 | 2.3 |
| 24.9063 | 3.57509 | 53.83 | 1.44 |
| 26.2145 | 3.39958 | 162.88 | 4.37 |
| 26.4967 | 3.36401 | 70.31 | 1.89 |
| 27.9121 | 3.19655 | 77.79 | 2.09 |
| 28.2518 | 3.15888 | 73.86 | 1.98 |
| 28.5038 | 3.12894 | 27.21 | 0.73 |
| 28.9245 | 3.08694 | 75.46 | 2.02 |
| 29.5494 | 3.02306 | 128.81 | 3.46 |
| 29.7403 | 3.00161 | 68 | 1.82 |
| 30.0238 | 2.9739 | 31.42 | 0.84 |
| 30.9064 | 2.89335 | 28.94 | 0.78 |
| 31.283 | 2.85701 | 32.42 | 0.87 |
| 32.581 | 2.74836 | 23.05 | 0.62 |
| 33.6981 | 2.65977 | 25.93 | 0.7 |

### Example 11

Crystalline, Form K material of ((S)-2-amino-3-(3,4-dihydroxyphenyl) propanoyl)-L-tyrosine was according to Procedure B using methyl isobutyl ketone as solvent. The XRPD pattern of crystalline Form K is shown in FIG. 11. Characteristic peaks include one or more of the peaks shown in Table 11. Crystalline Form K displayed a thermogravimetric analysis (TGA) profile showing a mass loss of about 17 wt. % between about 22° C. to about 203° C. (FIG. 12).

TABLE 11

| Pos. [°2θ] | d-spacing [Å] | Height [cts] | Rel. Int. [%] |
|---|---|---|---|
| 7.5174 | 11.75052 | 27.75 | 8.64 |
| 8.528 | 10.36019 | 287.92 | 89.62 |
| 11.0104 | 8.03594 | 54.46 | 16.95 |
| 13.3882 | 6.61358 | 69.73 | 21.7 |
| 15.3555 | 5.77045 | 321.27 | 100 |
| 16.2266 | 5.45805 | 17.56 | 5.46 |
| 18.818 | 4.71576 | 20.94 | 6.52 |
| 20.7713 | 4.27297 | 87.91 | 27.36 |
| 21.0464 | 4.21773 | 42.56 | 13.25 |
| 21.3404 | 4.16029 | 18.34 | 5.71 |
| 22.1374 | 4.01558 | 33.44 | 10.41 |
| 25.0883 | 3.54957 | 33.56 | 10.45 |
| 25.8373 | 3.44835 | 26.12 | 8.13 |
| 27.6958 | 3.22102 | 22.27 | 6.93 |

### Example 12

Crystalline, Form L material of ((S)-2-amino-3-(3,4-dihydroxyphenyl) propanoyl)-L-tyrosine was according to Procedure B using trifluorethanol as solvent. Crystalline Form L material was also prepared according to Procedure D using acetone or heptane as solvent. The XRPD pattern of crystalline Form L is shown in FIG. 11. Characteristic peaks include one or more of the peaks shown in Table 12.

TABLE 12

| Pos. [°2θ] | d-spacing [Å] | Height [cts] | Rel. Int. [%] |
|---|---|---|---|
| 7.6255 | 11.59376 | 54.19 | 8.84 |
| 7.9684 | 11.0956 | 98.83 | 16.13 |
| 8.7021 | 10.16168 | 612.76 | 100 |
| 11.0684 | 7.99395 | 86.47 | 14.11 |
| 13.4377 | 6.58934 | 102.57 | 16.74 |
| 14.7156 | 6.01988 | 23.99 | 3.92 |
| 15.4714 | 5.72747 | 569 | 92.86 |
| 16.1661 | 5.48286 | 36.34 | 5.93 |
| 19.2161 | 4.61514 | 62 | 10.12 |
| 19.5175 | 4.54455 | 14.52 | 2.37 |
| 19.7848 | 4.48374 | 13.42 | 2.19 |
| 20.8502 | 4.25698 | 188.42 | 30.75 |
| 21.2228 | 4.18306 | 13.08 | 2.14 |
| 21.5119 | 4.12749 | 103.77 | 16.93 |
| 21.6733 | 4.09713 | 52.24 | 8.53 |
| 22.2108 | 4.00249 | 78.15 | 12.75 |
| 22.5808 | 3.93448 | 19.06 | 3.11 |
| 22.947 | 3.87572 | 36.43 | 5.95 |
| 24.6585 | 3.60747 | 15.25 | 2.49 |
| 25.3083 | 3.51629 | 59.89 | 9.77 |
| 25.9225 | 3.4372 | 42.24 | 6.89 |
| 26.2418 | 3.39328 | 20.52 | 3.35 |
| 26.6882 | 3.3403 | 23.82 | 3.89 |
| 27.7846 | 3.21094 | 81.11 | 13.24 |
| 28.8782 | 3.09178 | 29.96 | 4.89 |
| 29.5768 | 3.02032 | 39.57 | 6.46 |
| 31.1275 | 2.87331 | 17.3 | 2.82 |
| 32.2969 | 2.77189 | 11.5 | 1.88 |
| 34.0901 | 2.63007 | 13.91 | 2.27 |
| 35.6279 | 2.52001 | 14.77 | 2.41 |
| 36.9822 | 2.43077 | 16.86 | 2.75 |
| 38.9703 | 2.31122 | 6.94 | 1.13 |

### Example 13

Crystalline, Form M material of ((S)-2-amino-3-(3,4-dihydroxyphenyl) propanoyl)-L-tyrosine was according to Procedure D using tetrahydrofuran as solvent. The XRPD pattern of crystalline Form M is shown in FIG. 11. Characteristic peaks include one or more of the peaks shown in Table 13. Crystalline Form M displayed a thermogravimetric analysis (TGA) profile showing a mass loss of about 17 wt. % between about 22° C. to about 203° C. (FIG. 13).

TABLE 13

| Pos. [°2θ] | d-spacing [Å] | Height [cts] | Rel. Int. [%] |
|---|---|---|---|
| 7.6273 | 11.59103 | 42.39 | 13.43 |
| 8.7082 | 10.15456 | 315.59 | 100 |
| 8.7863 | 10.06453 | 280.46 | 88.87 |
| 11.1012 | 7.97044 | 36.88 | 11.69 |
| 13.4907 | 6.56358 | 57.73 | 18.29 |
| 14.7614 | 5.99633 | 16.73 | 5.3 |
| 15.5117 | 5.71267 | 271.15 | 85.92 |
| 16.1892 | 5.47511 | 21.88 | 6.93 |
| 19.3358 | 4.59062 | 31.62 | 10.02 |
| 20.9239 | 4.24566 | 146.55 | 46.44 |
| 21.4985 | 4.13346 | 63.26 | 20.05 |
| 21.7851 | 4.07973 | 72.96 | 23.12 |
| 22.3339 | 3.9807 | 39.52 | 12.52 |
| 24.7873 | 3.59198 | 14.03 | 4.45 |
| 25.3273 | 3.51662 | 37.5 | 11.88 |
| 26.0726 | 3.41775 | 19.82 | 6.28 |
| 26.6494 | 3.34507 | 21.26 | 6.74 |
| 27.8291 | 3.20589 | 52.45 | 16.62 |
| 28.953 | 3.08396 | 11.12 | 3.52 |
| 29.7993 | 2.99828 | 26.5 | 8.4 |
| 34.0836 | 2.63056 | 8.81 | 2.79 |
| 37.003 | 2.42945 | 10.68 | 3.38 |

### Example 14

The XRPD pattern of crystalline Form O of ((S)-2-amino-3-(3,4-dihydroxyphenyl) propanoyl)-L-tyrosine is shown in FIG. 14. Characteristic peaks include one or more of the peaks shown in Table 14.

TABLE 14

| Pos. [°2θ] | d-spacing [Å] | Height [cts] | Rel. Int. [%] |
|---|---|---|---|
| 7.4956 | 11.79442 | 69.81 | 6.26 |
| 8.6717 | 10.19719 | 1114.82 | 100 |
| 11.1231 | 7.95478 | 88.88 | 7.97 |
| 11.3781 | 7.77063 | 24.79 | 2.22 |
| 13.5839 | 6.51877 | 90.6 | 8.13 |
| 14.0394 | 6.30825 | 41.74 | 3.74 |
| 14.6179 | 6.05988 | 47.6 | 4.27 |
| 15.3947 | 5.75584 | 628.41 | 56.37 |
| 15.5376 | 5.7032 | 317.07 | 28.44 |
| 16.3852 | 5.41004 | 26.44 | 2.37 |
| 18.4792 | 4.80146 | 39.46 | 3.54 |
| 18.8679 | 4.7034 | 83.12 | 7.46 |
| 19.9284 | 4.45544 | 20.25 | 1.82 |
| 20.4225 | 4.34515 | 16.57 | 1.49 |
| 20.9981 | 4.23081 | 192.79 | 17.29 |
| 21.3818 | 4.15576 | 241.15 | 21.63 |
| 21.6019 | 4.11392 | 122.62 | 11 |
| 22.3474 | 3.97834 | 77.33 | 6.94 |
| 22.8411 | 3.89344 | 39.03 | 3.5 |
| 24.1113 | 3.68808 | 18.08 | 1.62 |
| 24.8031 | 3.58973 | 22.57 | 2.02 |
| 25.5391 | 3.48793 | 81.2 | 7.28 |
| 25.9892 | 3.42569 | 41.48 | 3.72 |
| 26.2534 | 3.39462 | 61.8 | 5.54 |
| 27.1032 | 3.29009 | 26.68 | 2.39 |
| 27.688 | 3.22192 | 37.33 | 3.35 |
| 28.1882 | 3.16586 | 66.19 | 5.94 |
| 29.3739 | 3.04072 | 62.32 | 5.59 |
| 31.6621 | 2.826 | 6.82 | 0.61 |
| 33.618 | 2.66592 | 8.66 | 0.78 |
| 35.6376 | 2.51934 | 25.08 | 2.25 |

### Example 15

The XRPD pattern of crystalline Form P of ((S)-2-amino-3-(3,4-dihydroxyphenyl) propanoyl)-L-tyrosine is shown in FIG. 15. Characteristic peaks include one or more of the peaks shown in Table 15.

TABLE 15

| Pos. [°2θ] | d-spacing [Å] | Height [cts] | Rel. Int. [%] |
|---|---|---|---|
| 7.4415 | 11.87993 | 548.79 | 23.51 |
| 8.7392 | 10.11859 | 2334.51 | 100 |
| 10.492 | 8.43178 | 46.19 | 1.98 |
| 11.3523 | 7.79466 | 151.96 | 6.51 |
| 12.3947 | 7.14139 | 118.43 | 5.07 |
| 12.6856 | 6.9783 | 44.85 | 1.92 |
| 13.787 | 6.41785 | 29.98 | 1.28 |
| 13.957 | 6.34531 | 234.26 | 10.03 |
| 14.7053 | 6.02406 | 70.27 | 3.01 |
| 15.4923 | 5.7198 | 968.86 | 41.5 |
| 16.447 | 5.38987 | 48.78 | 2.09 |
| 17.418 | 5.0915 | 19.29 | 0.83 |
| 17.8517 | 4.9688 | 24.55 | 1.05 |
| 18.5399 | 4.78191 | 21.78 | 0.93 |
| 18.7085 | 4.7431 | 171.56 | 7.35 |
| 20.1959 | 4.39702 | 142.4 | 6.1 |
| 20.3494 | 4.3606 | 75.33 | 3.23 |
| 21.4278 | 4.14352 | 643.12 | 27.55 |
| 21.5185 | 4.12967 | 673.72 | 28.86 |
| 22.0352 | 4.03398 | 29.57 | 1.27 |
| 22.7931 | 3.90154 | 167.47 | 7.17 |
| 23.2705 | 3.82257 | 50.41 | 2.16 |
| 24.4696 | 3.63789 | 103.83 | 4.45 |
| 24.9263 | 3.57227 | 32.83 | 1.41 |
| 25.8441 | 3.44745 | 50.36 | 2.16 |
| 26.1866 | 3.40313 | 169.68 | 7.27 |
| 28.1885 | 3.16584 | 132.6 | 5.68 |
| 29.0089 | 3.07814 | 96.72 | 4.14 |
| 29.3734 | 3.04077 | 79.3 | 3.4 |
| 29.6847 | 3.00958 | 46.78 | 2 |
| 30.1256 | 2.96654 | 28.94 | 1.24 |
| 31.0579 | 2.87958 | 43.41 | 1.86 |
| 32.7825 | 2.73193 | 19.77 | 0.85 |
| 33.569 | 2.6697 | 42.24 | 1.81 |
| 34.1239 | 2.62755 | 27.93 | 1.2 |
| 34.4297 | 2.60275 | 22.42 | 0.96 |

### Example 16

The XRPD pattern of crystalline Form Q of ((S)-2-amino-3-(3,4-dihydroxyphenyl) propanoyl)-L-tyrosine is shown in FIG. 15. Characteristic peaks include one or more of the peaks shown in Table 16.

TABLE 16

| Pos. [°2θ] | d-spacing [Å] | Height [cts] | Rel. Int. [%] |
|---|---|---|---|
| 8.7176 | 10.14358 | 190.34 | 100 |
| 13.4782 | 6.56963 | 21.96 | 11.54 |
| 15.4477 | 5.73619 | 110.32 | 57.96 |
| 19.2316 | 4.61144 | 24.45 | 12.84 |
| 20.909 | 4.24865 | 70.84 | 37.22 |
| 21.5638 | 4.1211 | 45.63 | 23.97 |
| 22.2271 | 3.99628 | 22.88 | 12.02 |
| 25.387 | 3.50848 | 23.29 | 12.24 |
| 27.8029 | 3.20887 | 13.15 | 6.91 |
| 29.6549 | 3.01255 | 10.72 | 5.63 |

### Example 17

Amorphous material of ((S)-2-amino-3-(3,4-dihydroxyphenyl) propanoyl)-L-tyrosine was prepared as follows. A sample of crystalline Form N material of ((S)-2-amino-3-(3,4-dihydroxyphenyl) propanoyl)-L-tyrosine (400 mg) was dissolved in methanol (20 mL) with the help of sonication and heating and the solution filtered through a 0.2 μm PTFE filter. The filtrate was rotary evaporated under vacuum at 60° C. and 150 RPM for 15 minutes to remove the methanol solvent. A colourless film was observed. The flask was removed from the water bath and dried overnight under vacuum at room temperature. During this time, the film changed in appearance to consist of off-white solids. XRPD analysis indicated that the material was amorphous, as shown in FIG. 16.

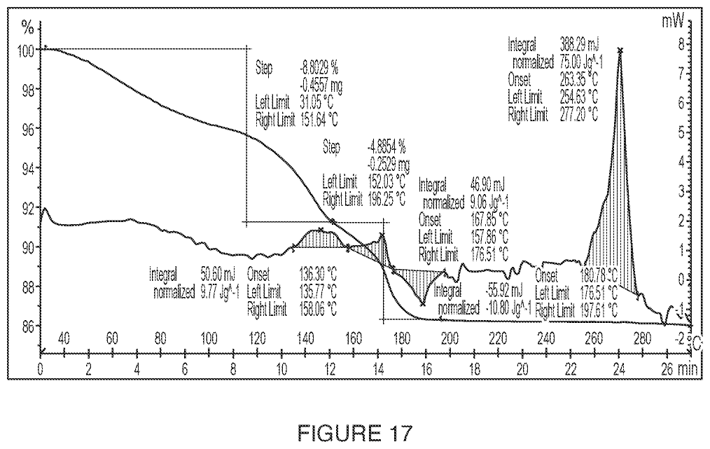

The amorphous form of ((S)-2-amino-3-(3,4-dihydroxyphenyl) propanoyl)-L-tyrosine displayed a thermogravimetric analysis (TGA) profile showing a mass loss of about 13.7 wt. % between about 42° C. to about 208° C. (FIG. 17). Karl Fischer (KF) analysis showed 0.96% water by weight (about 0.2 equivalent by molar ratio).

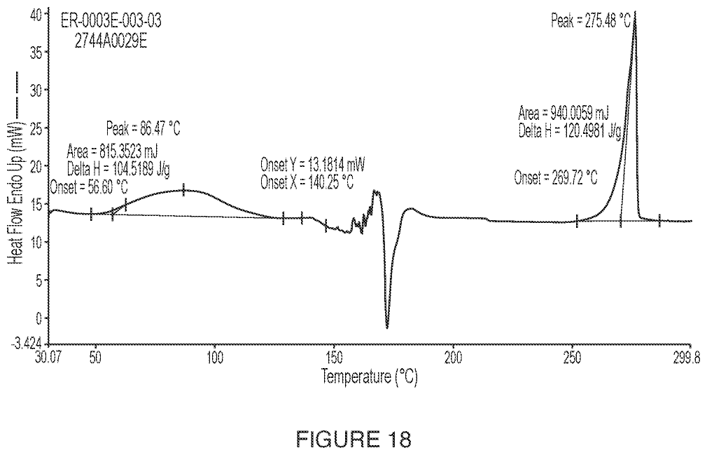

FIG. 18 depicts the differential scanning calorimetry (DSC) profile of the amorphous form. As shown in FIG. 18, after drying the amorphous material under vacuum for a second night, the amorphous form showed a characteristic broad endotherm with an onset of about 57° C. and a peak of about 86° C.; a characteristic exotherm with an onset of about 140° C. a characteristic exotherm with an onset of about 177° C.; and a characteristic endotherm with an onset of about 270° C. and a peak of about 275° C.

The amorphous form displayed a dynamic vapor sorption (DVS) profile showing a reversable total mass change of about 4.19 wt. % between about 40 to about 80% relative humidity (RH) at 25° C., indicating that the amorphous form is hygroscopic. Analysis by XRPD following DVS cycling showed that the amorphous form converted to Pattern N material during DVS analysis.

### Example 18a: Purification of ((S)-2-amino-3-(3,4-dihydroxyphenyl) propanoyl)-L-tyrosine-30% active carbon

20 gr of ((S)-2-amino-3-(3,4-dihydroxyphenyl) propanoyl)-L-tyrosine (polymorph N, having a color of BY3) was dissolved in 600 ml methanol. 6 gr of active carbon in dry form was added to the vessel, and the contents were mixed. After about 45 minutes the active carbon was filtered out. An additional amount of 6 gr of active carbon were added to the vessel and the contents were mixed. After about 45 minutes, the active carbon was filtered out. The solution was distilled to remove most of the methanol, leaving about 100 ml of methanol in the vessel, and then charged with 40 ml of water and 200 ml of iso-propyl alcohol (IPA). The contents of the vessel were stirred for about 2 hours, and subsequently filtered, providing a wet solid. An additional volume of 100 ml water, and the contents of the vessel were stirred for about 2 hours, after which the contents were filtered, the solid was collected and dried, thereby providing a highly pure form of ((S)-2-amino-3-(3,4-dihydroxyphenyl) propanoyl)-L-tyrosine (polymorph N).

The highly pure form of ((S)-2-amino-3-(3,4-dihydroxyphenyl) propanoyl)-L-tyrosine prepared according to the process, as detailed above, had a purity of 99.632%, measured by HPLC (1.0 RRT). The details of the HPLC results are provided in Table 17 below. LD-Tyr in the tables below corresponds to ((S)-2-amino-3-(3,4-dihydroxyphenyl) propanoyl)-L-tyrosine.

TABLE 17

| Name | RT | Area | % Area | RT Ratio |
|---|---|---|---|---|
| Levodopa | 6.38 | 493 | 0.005 | 0.27 |
| Peak2 | 19.18 | 2722 | 0.025 | 0.82 |
| Diketopiperazine | 20.99 | 512 | 0.005 | 0.90 |
| Peak4 | 22.45 | 6986 | 0.065 | 0.96 |
| LD-Tyr | 23.32 | 10702114 | 99.632 | 1.00 |
| Peak6 | 24.52 | 2560 | 0.024 | 1.05 |
| Peak7 | 26.61 | 843 | 0.008 | 1.14 |
| Peak8 | 27.19 | 1277 | 0.012 | 1.17 |
| Peak9 | 28.83 | 194 | 0.002 | 1.24 |
| Peak10 | 29.71 | 121 | 0.001 | 1.27 |
| Peak11 | 30.26 | 547 | 0.005 | 1.30 |
| Peak12 | 31.23 | 778 | 0.007 | 1.34 |
| Peak13 | 31.55 | 501 | 0.005 | 1.35 |
| Peak14 | 31.67 | 198 | 0.002 | 1.36 |
| Peak15 | 31.84 | 849 | 0.008 | 1.37 |
| Peak16 | 31.96 | 796 | 0.007 | 1.37 |
| Peak17 | 32.24 | 1032 | 0.010 | 1.38 |
| Peak18 | 32.44 | 781 | 0.007 | 1.39 |
| Peak19 | 32.77 | 226 | 0.002 | 1.41 |
| Peak20 | 32.90 | 688 | 0.006 | 1.41 |
| Peak21 | 33.18 | 428 | 0.004 | 1.42 |
| Peak22 | 33.39 | 1621 | 0.015 | 1.43 |
| Peak23 | 33.64 | 2883 | 0.027 | 1.44 |
| Peak24 | 34.03 | 78 | 0.001 | 1.46 |
| Peak25 | 34.33 | 371 | 0.003 | 1.47 |
| Peak26 | 34.67 | 627 | 0.006 | 1.49 |
| Peak27 | 34.91 | 220 | 0.002 | 1.50 |
| Peak28 | 35.08 | 170 | 0.002 | 1.50 |
| Peak29 | 36.73 | 197 | 0.002 | 1.58 |
| Peak30 | 37.24 | 1646 | 0.015 | 1.60 |
| Peak31 | 40.01 | 8889 | 0.083 | 1.72 |
| Peak32 | 40.46 | 297 | 0.003 | 1.74 |
| Sum | | 10741645 | | |

The HPLC was obtained with mobile phase A of 0.1% TFA in water and mobile phase B of 0.1% TFA in MeOH:ACN (8:2), with an injection volume of 20.00 μL, run time of 65.0 mins, diluent of 0.1% OPA in water, with column: Atlantis T3 150*4.6 mm 3μ, flow rate of 1.0 mL/min and sample concentration of 1.0 mg/mL.

The color of the highly pure form of ((S)-2-amino-3-(3,4-dihydroxyphenyl) propanoyl)-L-tyrosine prepared according to the process, as detailed above, was BY6. The yield was 65%.

### Example 18b: Purification of ((S)-2-amino-3-(3,4-dihydroxyphenyl) propanoyl)-L-tyrosine-15% active carbon, 20 gr sample

20 gr of ((S)-2-amino-3-(3,4-dihydroxyphenyl) propanoyl)-L-tyrosine (polymorph N, having a color of BY3) was dissolved in 600 ml methanol. 3 gr of active carbon in dry form was added to the vessel, and the contents were mixed. After about 45 minutes the active carbon was filtered out. An additional amount of 3 gr of active carbon were added to the vessel and the contents were mixed. After about 45 minutes, the active carbon was filtered out. The solution was distilled to remove most of the methanol, leaving about 100 ml of methanol in the vessel, and then charged with 40 ml of water and 200 ml of iso-propyl alcohol (IPA). The contents of the vessel were stirred for about 2 hours, and subsequently filtered, providing a wet solid. An additional volume of 100 ml water, and the contents of the vessel were stirred for about 2 hours, after which the contents were filtered, the solid was collected and dried, thereby providing a highly pure form of ((S)-2-amino-3-(3,4-dihydroxyphenyl) propanoyl)-L-tyrosine (polymorph N).

The highly pure form of ((S)-2-amino-3-(3,4-dihydroxyphenyl) propanoyl)-L-tyrosine prepared according to the process, as detailed above, had a purity of 99.517%, measured by HPLC (1.0 RRT). The details of the HPLC results are provided in Table 18 below.

The HPLC was obtained with mobile phase A of 0.1% TFA in water and mobile phase B of 0.1% TFA in MeOH:ACN (8:2), with an injection volume of 20.00 μL, run time of 65.0 mins, diluent of 0.1% OPA in water, with column: Atlantis T3 150*4.6 mm 3μ, flow rate of 1.0 mL/min and sample concentration of 1.0 mg/mL.

TABLE 18

| Name | RT | Area | % Area | RT Ratio |
|---|---|---|---|---|
| Peak1 | 4.03 | 426 | 0.002 | 0.18 |
| Levodopa | 6.14 | 734 | 0.004 | 0.27 |
| Peak3 | 19.05 | 5634 | 0.029 | 0.83 |
| Diketopiperazine | 21.15 | 967 | 0.005 | 0.92 |
| Peak5 | 22.25 | 19631 | 0.099 | 0.97 |
| LD-Tyr | 22.94 | 19664137 | 99.517 | 1.00 |
| Peak7 | 24.27 | 5663 | 0.029 | 1.06 |
| Peak8 | 26.45 | 1664 | 0.008 | 1.15 |
| Peak9 | 27.01 | 23100 | 0.117 | 1.18 |
| Peak10 | 28.73 | 672 | 0.003 | 1.25 |
| Peak11 | 29.72 | 1747 | 0.009 | 1.30 |
| Peak12 | 30.17 | 811 | 0.004 | 1.31 |
| Peak13 | 30.54 | 337 | 0.002 | 1.33 |
| Peak14 | 30.62 | 447 | 0.002 | 1.33 |
| Peak15 | 30.95 | 431 | 0.002 | 1.35 |
| Peak16 | 31.11 | 1408 | 0.007 | 1.36 |
| Peak17 | 31.33 | 1800 | 0.009 | 1.37 |
| Peak18 | 31.42 | 385 | 0.002 | 1.37 |
| Peak19 | 31.58 | 1930 | 0.010 | 1.38 |
| Peak20 | 31.71 | 324 | 0.002 | 1.38 |
| Peak21 | 31.85 | 1059 | 0.005 | 1.39 |
| Peak22 | 31.98 | 3350 | 0.017 | 1.39 |
| Peak23 | 32.31 | 1615 | 0.008 | 1.41 |
| Peak24 | 32.64 | 353 | 0.002 | 1.42 |
| Peak25 | 32.94 | 590 | 0.003 | 1.44 |
| Peak26 | 33.07 | 540 | 0.003 | 1.44 |
| Peak27 | 33.27 | 4362 | 0.022 | 1.45 |
| Peak28 | 33.52 | 8420 | 0.043 | 1.46 |
| Peak29 | 34.19 | 1169 | 0.006 | 1.49 |
| Peak30 | 34.57 | 968 | 0.005 | 1.51 |
| Peak31 | 34.78 | 314 | 0.002 | 1.52 |
| Peak32 | 34.97 | 290 | 0.001 | 1.52 |
| Peak33 | 36.51 | 420 | 0.002 | 1.59 |
| Peak34 | 37.00 | 3507 | 0.018 | 1.61 |
| Peak35 | 39.98 | 271 | 0.001 | 1.74 |
| Sum | | 19759477 | | |

The color of the highly pure form of ((S)-2-amino-3-(3,4-dihydroxyphenyl) propanoyl)-L-tyrosine prepared according to the process, as detailed above, was BY6. The yield was 82.5%. A comparison of the yield to that obtained in Example 18a shows that using 15% active carbon is preferable than 30% active carbon.

### Example 18c: Purification of ((S)-2-amino-3-(3,4-dihydroxyphenyl) propanoyl)-L-tyrosine-15% active carbon, 100 gr samples

100 gr of ((S)-2-amino-3-(3,4-dihydroxyphenyl) propanoyl)-L-tyrosine (polymorph N, having a color of BY3) was dissolved in 600 ml methanol. 15 gr of active carbon in dry form was added to the vessel, and the contents were mixed. After about 45 minutes the active carbon was filtered out. An additional amount of 15 gr of active carbon were added to the vessel and the contents were mixed. After about 45 minutes, the active carbon was filtered out. The solution was distilled to remove most of the methanol, leaving about 100 ml of methanol in the vessel, and then charged with 40 ml of water and 200 ml of iso-propyl alcohol (IPA). The contents of the vessel were stirred for about 2 hours, and subsequently filtered, providing a wet solid. An additional volume of 100 ml water, and the contents of the vessel were stirred for about 2 hours, after which the contents were filtered, the solid was collected and dried, thereby providing a highly pure form of ((S)-2-amino-3-(3,4-dihydroxyphenyl) propanoyl)-L-tyrosine (polymorph N). This test was run twice, on two separate 100 gr samples.

The highly pure forms of ((S)-2-amino-3-(3,4-dihydroxyphenyl) propanoyl)-L-tyrosine prepared according to the process, as detailed above, had purity levels of 99.56% and 99.688%, measured by HPLC (1.0 RRT). The details of the HPLC results are provided in Tables 19-1, 19-2, 20-1, and 20-2 below.

The HPLC was obtained with mobile phase A of 0.1% TFA in water and mobile phase B of 0.1% TFA in MeOH:ACN (8:2), with an injection volume of 20.00 μL, run time of 65.0 mins, diluent of 0.1% OPA in water, with column: Atlantis T3 150*4.6 mm 3μ, flow rate of 1.0 mL/min and sample concentration of 1.0 mg/mL.

TABLE 19-1

| | | | | |
|---|---|---|---|---|
| Name | RT | Area | % Area | RT Ratio |

(prep 1)

| | | | | |
|---|---|---|---|---|
| Levodopa | 5.81 | 721 | 0.01 | 0.26 |
| Diketopiperazine | 20.81 | 2045 | 0.02 | 0.92 |
| Peak 3 | 21.71 | 12630 | 0.10 | 0.96 |
| LD-Tyr | 22.56 | 12009893 | 99.81 | 1.00 |
| Peak 5 | 33.38 | 5649 | 0.05 | 1.48 |
| Z-L-Dopa | 36.88 | 2084 | 0.02 | 1.64 |
| Sum | | 12033024 | | |

(prep 2)

| | | | | |
|---|---|---|---|---|
| Levodopa | 5.80 | 738 | 0.01 | 0.26 |
| Diketopiperazine | 20.82 | 2298 | 0.02 | 0.92 |
| Peak 3 | 21.72 | 12777 | 0.11 | 0.96 |
| LD-Tyr | 22.56 | 12003815 | 99.81 | 1.00 |
| Peak 5 | 33.39 | 5614 | 0.05 | 1.48 |
| Z-L-Dopa | 36.89 | 2014 | 0.02 | 1.64 |
| Sum | | 12027256 | | |

TABLE 20-1

| | | | | |
|---|---|---|---|---|
| Name | RT | Area | % Area | RT Ratio |

(prep 1)

| | | | | |
|---|---|---|---|---|
| Levodopa | 5.80 | 742 | 0.01 | 0.26 |
| Diketopiperazine | 20.81 | 2435 | 0.02 | 0.92 |
| Peak 3 | 21.70 | 12751 | 0.11 | 0.96 |
| LD-Tyr | 22.55 | 11969584 | 99.81 | 1.00 |
| Peak 5 | 33.37 | 5351 | 0.04 | 1.48 |
| Z-L-Dopa | 36.87 | 2088 | 0.02 | 1.64 |
| Sum | | 11992951 | | |

(prep 2)

| | | | | |
|---|---|---|---|---|
| Levodopa | 5.80 | 731 | 0.01 | 0.26 |
| Diketopiperazine | 20.80 | 2540 | 0.02 | 0.92 |
| Peak 3 | 21.70 | 12900 | 0.11 | 0.96 |
| LD-Tyr | 22.54 | 11957202 | 99.80 | 1.00 |
| Peak 5 | 33.38 | 5334 | 0.04 | 1.48 |
| Z-L-Dopa | 36.88 | 2108 | 0.02 | 1.64 |
| Sum | | 11980815 | | |

The color of the highly pure form of ((S)-2-amino-3-(3,4-dihydroxyphenyl) propanoyl)-L-tyrosine prepared from both batches, according to the process as detailed above, was BY6. The yield was 77% and 78%, respectively for each batch. A comparison of the yields to those obtained in Examples 18a and 18b shows that (a) using 15% active carbon is preferable than 30% active carbon; and (b) the yield is more or less preserved, even when the purified batch is larger.

### Example 19: Preparation of highly pure ((S)-2-amino-3-(3,4-dihydroxyphenyl) propanoyl)-L-tyrosine

10 gr of Z-L-Dopa-Tyr-OBzl, structure of which is shown below,

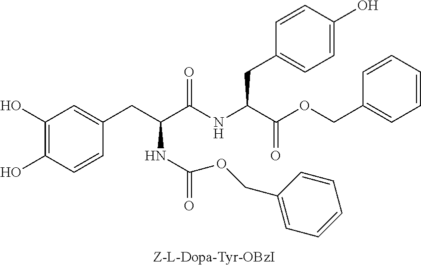 [SMILES: O=C(N[C@@H](Cc1ccc(O)c(O)c1)C(=O)N[C@@H](Cc1ccc(O)cc1)C(=O)OCc1ccccc1)OCc1ccccc1]

were mixed in a vessel together with 200 ml methanol and 1 gr of palladium on carbon (Pd/C). The contents were stirred for about 1 hour, after which the contents were filtered, thereby removing the Pd/C. 2 gr of active carbon were added to the vessel, the contents were stirred for about 30 minutes, and subsequently filtered, thereby removing the active carbon. 1 gr of a palladium scavenger was added to the vessel, the contents were stirred for about 1 hour, and subsequently filtered, thereby removing the palladium scavenger. An additional amount of 2 gr of active carbon were added to the vessel, the contents were stirred for about 30 minutes, and subsequently filtered, thereby removing the active carbon. The remaining liquid in the vessel was distilled to remove most the methanol, leaving about 50 ml methanol in the vessel. The vessel was subsequently charged with 20 ml water and 100 ml iso-propyl alcohol (IPA). The solution was stirred for about 2 hours, after which it was filtered, and the solid was collected.

The obtained solid was dissolved in 50 ml water, stirred for about 2 hours, filtered and dried, thereby providing a highly pure polymorph N form of ((S)-2-amino-3-(3,4-dihydroxyphenyl) propanoyl)-L-tyrosine.

The highly pure form of ((S)-2-amino-3-(3,4-dihydroxyphenyl) propanoyl)-L-tyrosine prepared according to the process, as detailed above, had a purity of 99.191%, measured by HPLC (1.0 RRT). The details of the HPLC results are provided in Table 21 below:

TABLE 21

| Name | RT | Area | % Area | RT Ratio |
|---|---|---|---|---|
| Peak1 | 3.90 | 363 | 0.003 | 0.17 |
| Levodopa | 5.91 | 401 | 0.004 | 0.26 |
| Peak3 | 16.86 | 1508 | 0.014 | 0.74 |
| Peak4 | 18.45 | 3271 | 0.030 | 0.81 |
| Diketopiperazine | 20.71 | 645 | 0.006 | 0.91 |
| Peak6 | 21.79 | 16462 | 0.151 | 0.96 |
| LD-Tyr | 22.65 | 10796129 | 99.191 | 1.00 |
| Peak8 | 23.90 | 4703 | 0.043 | 1.06 |
| Peak9 | 25.99 | 1195 | 0.011 | 1.15 |
| Peak10 | 26.62 | 1416 | 0.013 | 1.18 |
| Peak11 | 29.31 | 906 | 0.008 | 1.29 |
| Peak12 | 29.59 | 336 | 0.003 | 1.31 |
| Peak13 | 29.93 | 638 | 0.006 | 1.32 |
| Peak14 | 30.39 | 1438 | 0.013 | 1.34 |
| Peak15 | 30.83 | 2183 | 0.020 | 1.36 |
| Peak16 | 31.22 | 730 | 0.007 | 1.38 |
| Peak17 | 31.48 | 952 | 0.009 | 1.39 |
| Peak18 | 31.73 | 88 | 0.001 | 1.40 |
| Peak19 | 31.77 | 165 | 0.002 | 1.40 |
| Peak20 | 31.90 | 1510 | 0.014 | 1.41 |
| Peak21 | 32.20 | 1331 | 0.012 | 1.42 |
| Peak22 | 32.54 | 87 | 0.001 | 1.44 |
| Peak23 | 32.76 | 1074 | 0.010 | 1.45 |
| Peak24 | 32.97 | 399 | 0.004 | 1.46 |
| Peak25 | 33.17 | 579 | 0.005 | 1.46 |
| Peak26 | 33.43 | 3091 | 0.028 | 1.48 |
| Peak27 | 33.83 | 185 | 0.002 | 1.49 |
| Peak28 | 34.12 | 366 | 0.003 | 1.51 |
| Peak29 | 34.36 | 72 | 0.001 | 1.52 |
| Peak30 | 34.49 | 814 | 0.007 | 1.52 |
| Peak31 | 34.73 | 223 | 0.002 | 1.53 |
| Peak32 | 34.95 | 5315 | 0.049 | 1.54 |
| Peak33 | 35.30 | 253 | 0.002 | 1.56 |
| H-Tyr-OBZ1 | 35.75 | 249 | 0.002 | 1.58 |
| Peak35 | 35.84 | 270 | 0.002 | 1.58 |
| Peak36 | 36.51 | 486 | 0.004 | 1.61 |
| Peak37 | 37.01 | 1730 | 0.016 | 1.63 |
| Peak38 | 37.94 | 173 | 0.002 | 1.68 |
| Peak39 | 39.37 | 363 | 0.003 | 1.74 |
| Peak40 | 39.74 | 41 | 0.000 | 1.75 |
| Peak41 | 39.92 | 31677 | 0.291 | 1.76 |
| Peak 42 | 40.77 | 336 | 0.003 | 1.80 |
| Sum | | 10884153 | | |

The HPLC was obtained with mobile phase A of 0.1% TFA in water and mobile phase B of 0.1% TFA in MeOH:ACN (8:2), with an injection volume of 20.00 μL, run time of 65.0 mins, diluent of 0.1% OPA in water, with column: Atlantis T3 150*4.6 mm 3μ, flow rate of 1.0 mL/min and sample concentration of 1.0 mg/mL.

The color of the highly pure form of ((S)-2-amino-3-(3,4-dihydroxyphenyl) propanoyl)-L-tyrosine prepared according to the process, as detailed above, was B6.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A crystalline form of ((S)-2-amino-3-(3,4-dihydroxyphenyl) propanoyl)-L-tyrosine (Form N), characterized by a powder X-ray diffraction pattern having a characteristic peak in degrees 2θ at about 20.1.

2. The crystalline form of claim 1, characterized by a powder X-ray diffraction pattern having characteristic peaks in degrees 2θ at about 12.6, 16.3, and 20.1.

3. The crystalline form of claim 1 or 2, characterized by a powder X-ray diffraction pattern having characteristic peaks in degrees 2θ at about 7.7, 8.7, 12.6, 16.3, 20.1, and 25.7.

4. The crystalline form of any one of claim 1-3, characterized by a powder X-ray diffraction pattern having characteristic peaks in degrees 2θ at about 7.7, 8.7, 8.8, 12.6, 16.2, 16.3, 17.3, 20.1, 25.7, 29.8, 30.0, and 34.2.

5. The crystalline form of any one of claims 1-4, wherein the powder X-ray diffraction pattern was obtained using Cu Kα radiation.

6. The crystalline form of any one of claims 1-5, characterized by a differential scanning calorimetry (DSC) profile having a characteristic endotherm with an onset of about 135° C. and a peak of about 159° C.; a characteristic endotherm with an onset of about 174° C. and a peak of about 176° C.; a characteristic exotherm with an onset of about 177° C.; and a peak of about 178° C.; and a characteristic endotherm with an onset of about 272° C. and a peak of about 278° C.; and a thermogravimetric analysis (TGA) profile showing a mass loss of about 15 wt. % between about 22° C. to about 196° C.

7. A crystalline form of ((S)-2-amino-3-(3,4-dihydroxyphenyl) propanoyl)-L-tyrosine (Form A), characterized by a powder X-ray diffraction pattern having a characteristic peak in degrees 2θ at about 4.7, 9.5, 18.3, 19.0, and 21.1, wherein the powder X-ray diffraction pattern was obtained using Cu Kα radiation.

8. The crystalline form of claim 8, characterized by a thermogravimetric analysis (TGA) profile showing a mass loss of about 10.2 wt. % between about 40° C. to about 200° C.

9. A crystalline form of ((S)-2-amino-3-(3,4-dihydroxyphenyl) propanoyl)-L-tyrosine (Form B), characterized by a powder X-ray diffraction pattern having a characteristic peak in degrees 2θ at about 14.5, 14.6, 14.8, 17.7, 20.3, 22.9, 23.2, 25.4, 27.3, 29.5, 30.2, and 31.3, wherein the powder X-ray diffraction pattern was obtained using Cu Kα radiation.

10. The crystalline form of claim 9, characterized by a differential scanning calorimetry profile having a characteristic endotherm with an onset of about 96° C. and a peak of about 119° C.; a characteristic endotherm with an onset of about 161° C. and a peak of about 176° C.; and a characteristic endotherm with an onset of about 275° C. and a peak of about 280° C.; and a thermogravimetric analysis (TGA) profile showing a mass loss of about 15.6 wt. % between about 40° C. to about 200° C.

11. A crystalline form of ((S)-2-amino-3-(3,4-dihydroxyphenyl) propanoyl)-L-tyrosine (Form C), characterized by a powder X-ray diffraction pattern having a characteristic peak in degrees 2θ at about 7.7, 9.0, 13.3, 13.5, 15.2, 15.4, 19.7, 20.5, 22.1, 25.7, 27.9, and 28.1, wherein the powder X-ray diffraction pattern was obtained using Cu Kα radiation.

12. The crystalline form of claim 11, characterized by a differential scanning calorimetry profile having a characteristic endotherm with an onset of about 145° C. and a peak of about 158° C.; a characteristic exotherm with an onset of about 175° C. and a peak of about 178° C.; and a characteristic endotherm with an onset of about 269° C. and a peak of about 275° C., and a thermogravimetric analysis (TGA) profile showing a mass loss of about 1.75 wt. % between about 30° C. to about 190° C.

13. A crystalline form of ((S)-2-amino-3-(3,4-dihydroxyphenyl) propanoyl)-L-tyrosine (Form D), characterized by a powder X-ray diffraction pattern having a characteristic peak in degrees 2θ at about 11.7, 17.0, 17.8, 18.6, 18.9, 19.2, 20.9, 23.6, 26.1, 26.7, 27.3, and 28.5, wherein the powder X-ray diffraction pattern was obtained using Cu Kα radiation.

14. The crystalline form of claim 13, characterized by a differential scanning calorimetry profile having a characteristic endotherm with an onset of about 79° C. and a peak of about 97° C.; a characteristic endotherm with an onset of about 147° C. and a peak of about 152° C.; a characteristic endotherm with an onset of about 175° C. and a peak of about 176° C.; a characteristic exotherm with an onset of about 178° C.; and a peak of about 179° C.; a characteristic exotherm with an onset of about 183° C. and a peak of about 191° C.; and a characteristic endotherm with an onset of about 263° C. and a peak of about 270° C.; and a thermogravimetric analysis (TGA) profile showing a mass loss of about 38 wt. % between about 30° C. to about 211° C.

15. A crystalline form of ((S)-2-amino-3-(3,4-dihydroxyphenyl) propanoyl)-L-tyrosine (Form E), characterized by a powder X-ray diffraction pattern having a characteristic peak in degrees 2θ at about 10.8, 12.5, 14.8, 15.2, 17.9, 20.3, 20.9, 23.3, 23.5, 24.4, 25.8, and 26.3, wherein the powder X-ray diffraction pattern was obtained using Cu Kα radiation.

16. The crystalline form of claim 15, characterized by a thermogravimetric analysis (TGA) profile showing a mass loss of about 11.5 wt. % between about 40° C. to about 200° C.

17. A crystalline form of ((S)-2-amino-3-(3,4-dihydroxyphenyl) propanoyl)-L-tyrosine (Form G), characterized by a powder X-ray diffraction pattern having a characteristic peak in degrees 2θ at about 7.6, 8.8, 13.5, 15.3, 15.5, 19.4, 21.0, 21.8, 22.3, 25.4, 27.8, and 29.9, wherein the powder X-ray diffraction pattern was obtained using Cu Kα radiation.

18. The crystalline form of claim 17, characterized by a differential scanning calorimetry profile having a characteristic endotherm with an onset of about 126° C. and a peak of about 130° C.; a characteristic endotherm with an onset of about 159° C. and a peak of about 169° C.; a characteristic exotherm with an onset of about 171° C.; and a peak of about 176° C.; and a characteristic endotherm with an onset of about 264° C. and a peak of about 271° C.

19. A crystalline form of ((S)-2-amino-3-(3,4-dihydroxyphenyl) propanoyl)-L-tyrosine (Form H), characterized by a powder X-ray diffraction pattern having a characteristic peak in degrees 2θ at about 8.0, 8.7, 11.4, 12.3, 14.2, 14.6, 15.5, 16.1, 18.7, 21.1, 21.4, and 22.9, wherein the powder X-ray diffraction pattern was obtained using Cu Kα radiation.

20. A crystalline form of ((S)-2-amino-3-(3,4-dihydroxyphenyl) propanoyl)-L-tyrosine (Form I), characterized by a powder X-ray diffraction pattern having a characteristic peak in degrees 2θ at about 7.4, 7.5, 8.7, 8.8, 13.9, 14.2, 15.5, 21.1, 21.6, 21.9, 22.7, and 26.2, wherein the powder X-ray diffraction pattern was obtained using Cu Kα radiation.

21. The crystalline form of claim 20, characterized by a thermogravimetric analysis (TGA) profile showing a mass loss of about 11 wt. % between about 30° C. to about 212° C.

22. A crystalline form of ((S)-2-amino-3-(3,4-dihydroxyphenyl) propanoyl)-L-tyrosine (Form J), characterized by a powder X-ray diffraction pattern having a characteristic peak in degrees 2θ at about 4.4, 7.5, 8.8, 13.9, 15.5, 18.9, 21.4, 21.6, 21.8, 22.7, 26.2, and 29.5, wherein the powder X-ray diffraction pattern was obtained using Cu Kα radiation.

23. A crystalline form of ((S)-2-amino-3-(3,4-dihydroxyphenyl) propanoyl)-L-tyrosine (Form K), characterized by a powder X-ray diffraction pattern having a characteristic peak in degrees 2θ at about 7.5, 8.5, 11.0, 13.4, 15.4, 18.8, 20.8, 21.0, 22.1, 25.1, 25.8, and 27.7, wherein the powder X-ray diffraction pattern was obtained using Cu Kα radiation.

24. The crystalline form of claim 23, characterized by a thermogravimetric analysis (TGA) profile showing a mass loss of about 17 wt. % between about 22° C. to about 203° C.

25. A crystalline form of ((S)-2-amino-3-(3,4-dihydroxyphenyl) propanoyl)-L-tyrosine (Form L), characterized by a powder X-ray diffraction pattern having a characteristic peak in degrees 2θ at about 7.6, 8.0, 8.7, 11.1, 13.4, 15.5, 19.2, 20.9, 21.5, 22.2, 25.3, and 27.8, wherein the powder X-ray diffraction pattern was obtained using Cu Kα radiation.

26. A crystalline form of ((S)-2-amino-3-(3,4-dihydroxyphenyl) propanoyl)-L-tyrosine (Form M), characterized by a powder X-ray diffraction pattern having a characteristic peak in degrees 2θ at about 7.6, 8.7, 8.8, 11.1, 13.5, 15.5, 20.9, 21.5, 21.8, 22.3, 25.3, and 27.8, wherein the powder X-ray diffraction pattern was obtained using Cu Kα radiation.

27. The crystalline form of claim 26, characterized by a thermogravimetric analysis (TGA) profile showing a mass loss of about 17 wt. % between about 22° C. to about 199° C.

28. A crystalline form of ((S)-2-amino-3-(3,4-dihydroxyphenyl) propanoyl)-L-tyrosine (Form O), characterized by a powder X-ray diffraction pattern having a characteristic peak in degrees 2θ at about 7.5, 8.7, 11.1, 13.6, 15.4, 15.5, 18.9, 21.0, 21.4, 21.6, 22.3, and 25.5, wherein the powder X-ray diffraction pattern was obtained using Cu Kα radiation.

29. A crystalline form of ((S)-2-amino-3-(3,4-dihydroxyphenyl) propanoyl)-L-tyrosine (Form P), characterized by a powder X-ray diffraction pattern having a characteristic peak in degrees 2θ at about 7.4, 8.7, 11.4, 14.0, 15.5, 18.7, 20.2, 21.4, 21.5, 22.8, 26.2, and 28.2, wherein the powder X-ray diffraction pattern was obtained using Cu Kα radiation.

30. A crystalline form of ((S)-2-amino-3-(3,4-dihydroxyphenyl) propanoyl)-L-tyrosine (Form Q), characterized by a powder X-ray diffraction pattern having a characteristic peak in degrees 2θ at about 8.7, 13.5, 15.4, 19.2, 20.9, 21.6, 22.2, 25.4, 27.8, and 29.7, the powder X-ray diffraction pattern was obtained using Cu Kα radiation.

31. A pharmaceutical composition comprising a crystalline form of any one of claims 1-30, and a pharmaceutically acceptable excipient.

32. A pharmaceutical composition comprising the crystalline Form N of any one of claims 1-6, and a pharmaceutically acceptable excipient.

33. A pharmaceutical composition formed from the crystalline form of any one of claims 1-30.

34. A pharmaceutical composition formed from the crystalline Form N of any one of claims 1-6.

35. A drug substance comprising at least a detectable amount of the crystalline form of any one of claims 1-30.

36. A drug substance comprising a substantially pure crystalline form of any one of claims 1-30.

37. A drug substance comprising a substantially pure crystalline Form N of any one of claims 1-6.

38. The crystalline form according to any one of claims 1-30, having a purity of above about 99%.

39. The crystalline form according to any one of claims 1-30, having a purity of above about 99.5%.

40. The crystalline form according to any one of claims 1-30, having a purity of above about 99.6%.

41. The crystalline form according to any one of claims 1-30, having a purity of above about 99.9%.

42. The crystalline form according to claim 38, characterized by an HPLC chromatogram having a peak at about 22.65 RT.

43. The crystalline form according to claim 38, characterized by an HPLC chromatogram having peaks at about 16.86, 18.45, 21.79, 22.65, 23.90, 25.99, 26.62, 30.39, 30.83, 31.90, 32.20, 32.76, 33.43, 34.95, 37.01, and 39.92 RT.

44. The crystalline form according to claim 39, characterized by an HPLC chromatogram having a peak at about 22.56 RT.

45. The crystalline form according to claim 39, characterized by an HPLC chromatogram having peaks at about 5.81, 20.81, 21.71, 22.56, 33.38, and 36.88 RT.

46. The crystalline form according to claim 39, characterized by an HPLC chromatogram having a peak at about 22.56 RT.

47. The crystalline form according to claim 39, characterized by an HPLC chromatogram having peaks at about 5.80, 20.82, 21.72, 22.56, 33.39, and 36.89 RT.

48. The crystalline form according to claim 40, characterized by an HPLC chromatogram having a peak at about 22.55 RT.

49. The crystalline form according to claim 40, characterized by an HPLC chromatogram having peaks at about 5.80, 20.81, 21.70, 22.55, 33.37, and 36.87 RT.

50. The crystalline form according to claim 40, characterized by an HPLC chromatogram having a peak at about 22.54 RT.

51. The crystalline form according to claim 40, characterized by an HPLC chromatogram having peaks at about 5.80, 20.80, 21.70, 22.54, 33.38, and 36.88 RT.

52. The crystalline form according to any one of claims 38-51, which is crystalline form N.

53. The crystalline form according to any one of the previous claims, wherein the color of the crystalline form, when dissolved in TRIS buffer, is defined as Y5, Y6, Y7, B5, B6, B7, BY5, BY6 or BY7 according to the Color Reference Solutions Y1-Y7, relating to Ph. Eur. Y1-Y7 Certipur®, BY1-BY7, relating to Ph. Eur. BY1-BY7 Certipur®, and B1-B7, relating to Ph. Eur. B1-B7 Certipur®.

54. The crystalline form according to any one of claims 38-51, which is purified from an already prepared batch of crystalline form N.

55. The crystalline form according to any one of claims 38-51, wherein the purification process is performed during the preparation of crystalline form N from

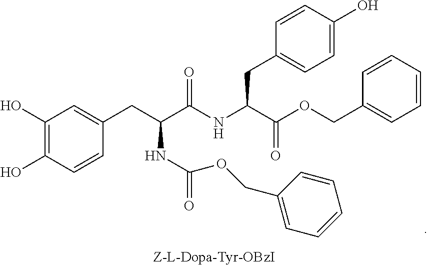 [SMILES: O=C(N[C@@H](Cc1ccc(O)c(O)c1)C(=O)N[C@@H](Cc1ccc(O)cc1)C(=O)OCc1ccccc1)OCc1ccccc1]

56. A process for purifying the crystalline form according to any one of the previous claims, wherein said process comprises use of active carbon.

57. The process according to claim 56, wherein the active carbon is selected from newcarb, pencarb, Alquandracarb, Eno PC Carb, HW Carb, Tri Carb, C-CA Activated carbon, C-HA Activated carbon, C-VA Activated carbon, C-VW Activated carbon, norite carbon, penta carb, or any combination thereof.

58. The process according to claim 56 or 57, wherein at least two portions of active carbon are used in the process.

59. The process according to any one of claims 56-58, wherein the amount of active carbon is between about 10% w/w to about 50% w/w, compared to the weight of the crystalline form being purified.

60. The process according to claim 59, wherein the amount of active carbon is about 15% w/w, compared to the weight of the crystalline form being purified.

61. The process according to any one of claims 56-60, further comprising the use of an acidic agent.

62. The process according to claim 61, wherein the acidic agent is selected from HF, HBr, trifluoroacetic acid and thioanisole, and BBr3, BCl3, or BF3 etherates, or a combination thereof.

63. The process according to claim 62, wherein the HBr is in acetic acid.

64. The process according to any one of claims 56-63, wherein the crystalline form is purified in an alcohol.

65. The process according to claim 64, wherein the alcohol is methanol, ethanol, propanol, iso-propanol, butanol, iso-butanol, or any other appropriate alcohol.

66. The process according to claim 64 or 65, wherein the alcohol is methanol.
